(19) United States
(12) Patent Application Publication
(10) Pub. No.: US 2025/0194904 A1
(43) Pub. Date: Jun. 19, 2025

(54) EXTERNAL WORKING CHANNELS FOR ENDOSCOPIC DEVICES

(71) Applicant: Neptune Medical Inc., Burlingame, CA (US)

(72) Inventors: Alexander Q. TILSON, Burlingame, CA (US); Stephan T. HOFFMANN, Danville, CA (US); Patricia HAYES-DANITZ, Menlo Park, CA (US); Kyle IMATANI, Mountain View, CA (US); Andrew MORRIS, San Francisco, CA (US); William EVANS, San Francisco, CA (US); Thomas HSIU, Palo Alto, CA (US); Kassidy MacPHAIL, San Francisco, CA (US)

(21) Appl. No.: 18/867,004

(22) PCT Filed: May 16, 2023
(86) PCT No.: PCT/US2023/067072

§ 371 Date: Nov. 18, 2024

Related U.S. Application Data
(60) Provisional application No. 63/342,618, filed on May 16, 2022.

(51) Int. Cl. A61B 1/00

(52) U.S. Cl. CPC A61B 1/00135; A61B 1/00078; A61B 1/00098

(57) ABSTRACT

Apparatuses (e.g., devices, systems, etc.) and methods that may provide access to one or more tools to a remote site in the body including expandable and external working channels that may be part of a tube that is coupled to an outer surface of an elongate medical device, such as an endoscope.

### CLAIM OF PRIORITY

This patent application claims priority to U.S. provisional patent application No. 63/342,618, entitled “EXTERNAL WORKING CHANNELS FOR ENDOSCOPIC DEVICES,” filed on May 16, 2022.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

The invention of endoscopy, the ability to see inside the body with a medical instrument, was considered revolutionary. The addition of working channels to endoscopes was an important improvement to the endoscope: a working channel can be used to insert a tool through the endoscope's proximal end, have it travel through the lumen through the body of the endoscope (and through the patient's anatomy), and emerge at the distal end of the endoscope, at the patient's target location. As a tool emerges from the endoscope, it can be used for a many different clinically relevant actions, including, for example: biopsy, grasping, manipulating, cutting, snaring, suturing, spraying, suctioning, clipping, or applying a treatment to tissue, that can include heat, cold, energy, RF, or radiation.

Flexible endoscopy further expanded the clinical utility, as it enabled the interrogation of tortuous anatomy.

As endoscopists tried to do more (“interventional endoscopy”), double working channel endoscopes were created. These proved to be much less effective than expected, as they increased girth (and therefore stiffness), still only allowed one more working channel, and also proved kinematically limited, as both lumens were typically parallel and central. The resulting tool exit trajectory from such systems is inherently kinematically limiting.

External working channels have been proposed as well, potentially offering multiple advantages. However, rigid external channels add considerable girth, and are either limited in number and therefore modest in utility, or larger in number and often not used, while being commensurately large. External working channels also typically increase system stiffness, which adversely impacts navigability and access. Currently available external channels are typically single channel, short and straight i.e., applied only on rigid scopes.

In general, there is a need for increased working channel diameters and/or an increase in the number of working channels. Increased working channel diameters enable larger tools, which can be more complex, stronger, easier to build, and can have increased clinical utility. Larger tools for suctioning and spraying can simply accomplish more, faster. As endoscopic procedures become more advanced, the use of multiple working channels may be particularly beneficial. Whereas most endoscopes have a single working channel, and some have two, there could be high clinical utility to have three, four, five, or even more working channels.

As endoscopes are used for increasingly complex procedures, there is also a need for a working channel that can be moved or actuated relative to the endoscope. A working channel that could be moved independently from the endoscope, including by being incorporated into a separate device (for example, an overtube) would allow additional levels of kinematic control: for example, independent axial control, the ability to be positioned in different radial positions, and the ability to remain stable while the bending section of the endoscope is angulated.

It is advantageous to have working channels that function over long lengths and amongst high tortuosity while maintaining low advance force and minimizing the adverse effect of capstan drag.

It may also be advantageous to be able to deploy multiple tools (including large diameter tools) through working channels, without disturbing the shape of the endoscope and how it is positioned in the anatomy, and ensuring a more consistent distal emergence at the site of care. This may be particularly helpful in devices (e.g., endoscopes) that may be selectively rigidized during use. These apparatuses may be referred to has dynamically rigidizing devices.

Described herein are external working channel apparatuses (e.g., devices, systems, etc.) that may address this shortcomings and needs.

### SUMMARY OF THE DISCLOSURE

In general, described herein are apparatuses (e.g., instruments, tools, devices, systems, etc.) and methods that may provide access to one or more tools to a remote site in the body. In particular, described herein are working channel sleeve apparatuses (device, system, etc.) that may include an inner tube (tubular region) configured to fit over an elongate medical device, and one or more (e.g., a plurality of) external working channels along the length of the internal tube. Any of these external working channel sleeve apparatuses may also be referred to equivalently as working channel sleeves, external working channel sleeves, or the like. Any of these working channel sleeves may include a core portion configured to surround the elongate member (e.g., catheter, endoscope, overtube, etc.) and one or more expandable working channels coupled to the core portion and/or formed integrally with the core portion. These working channels may be extensible, deflectable, elastic and/or lubricous. The working channel(s) may be formed of one or more filaments, including in particular a non-elastic filament. In some examples the working channel(s) may be formed as a knitted, braided, and/or woven structure. In some examples, the working channel(s) may be sewn or bonded. In some examples, the working channel may be formed of one or more laminated sheets of material. The material forming the working channels and core region (e.g., the elongate tubular body of the working channel sleeve) may be formed of one or more filaments, fibers, or wires.

As mentioned, the materials forming the working channel may be knitted, braided, woven or laminated. In some examples the material is formed of elements that do not cross. The materials may be precisely oriented, or they may be of a comparatively random orientation. In some examples, the working channels may be expandible working channels and may be formed as part a tubular device and may be coupled to an outer surface of an elongate medical device, e.g., a flexible, elongate member, such as a catheter, an overtube or an endoscope.

In some examples, described herein are braided, knitted or woven (and in particular knitted) working channel sleeves. The knitted working channel sleeves described herein may be similar to and may improve on the layflat working channels described in international (e.g., PCT patent publication number WO2021/242884 A1), herein incorporated by reference in its entirety.

For example, the working channel sleeves described herein include a core region, such as a central tube (e.g., a sleeve, sock, or cover portion) that may be formed of a material that is the same as or different from the material forming the working channel portion. This material may be elastic, non-elastic, and/or lubricious. In some examples one or more external working channels may be formed on (e.g., woven into) the central tube so that the entire working channel sleeve may be worn over and/or affixed to an elongate medical device, including a highly flexible medical device. In some examples the medical device may be part of the same system as the working channel sleeve. In some cases the working channel sleeve may be permanently or semi-permanently, or removably attached to the elongate medical device.

As mentioned, either or both the elongate tubular body and the external working channels may be formed of a filament or filaments (or bundle of filament) that may be knitted, woven or braided. Thus, the external and expandable working channels described herein may be knitted, braided, woven, sewn, or laminated, and may be manufactured from multiple parts that are assembled together, or they may come off a piece of automated and computer-controlled equipment in substantially complete form.

The working channel sleeves described herein may be attached, mounted, or otherwise combined with an elongate flexible member/medical device (e.g., catheter, endoscope, overtube, etc.). The tubular body may be attached to the outer surface of the elongate medical device, either at discrete locations along the length of the elongate medical device, e.g., the distal end region and the proximal end region, every 1 mm (or every 2 mm, or every 3 mm, or every 4 mm, every 5 mm, ever 10 mm, every 15 mm, every 20 mm, etc.), or continuously along the entire length of the inner tube (tubular body).

In some examples the external working channels described herein may be configured to easily allow the relative sliding of adjacent devices, materials, and anatomy. In some examples, the working channel sleeve apparatuses herein may be configured to allow the precise termination of exits from the working channel(s). For example, the working channels described herein may be configured to direct one or more apparatuses leaving the working channel in a predefined direction (e.g., radially inward, radially outward, etc.). Any of the apparatuses described herein may be configured so that a device may be easily inserted into the working channel proximally.

In any of the working channel sleeve apparatuses described herein the one or more external working channels may be internally configured to allow a tool to enter the working channel from a small aperture, including through a small incision or orifice. In some examples, the external working channel may be configured to have a small initial profile, and only expand as-needed. In any of the working channel sleeve apparatuses descried herein the external working channels may be pre-installed as part of the device. Alternatively, the working channel sleeve may be attached or installed by the user onto the endoscope or other elongate member. The working channels may be configured to be lubricious and elastic and may therefore be configured to allow easier sliding of tools within the working channel(s). As tortuosity increases, capstan drag equations typically dictate exponential decay in device performance, the elastic and lubricous working channels described herein may allow tools inserted into the working channel(s) to slide with the lowest possible drag. Similarly, the capstan drag equation may dictate the ease of endoscope sliding relative to anatomy, and the elastic and lubricous working channels described herein may be configured to provide the lowest possible drag relative to a patient's anatomy.

The working channel sleeves, including either or both the tubular body and/or the external working channels described herein formed of an elastic and lubricous material may be used with an endoscope. In general, the methods and apparatuses described herein may be used with endoscopes adapted for use across a wide range of endoscopic procedures, including colonoscopy, enteroscopy, esophagogastroduodenoscopy (EGD), enteroscopy, endoscopic retrograde cholangiopancreatography (ERCP), interventional endoscopy procedures (including ESD (Endoscopic Submucosal Dissection) and EMR (Endoscopic Mucosal Resection)), robotic flexible endoscopy, trans-oral robotic surgery (TORS), altered anatomy cases (including Roux-en-Y), and during NOTES (Natural Orifice Transluminal Endoscopic Surgery) procedures. Any of the methods and apparatuses described herein may be configured for use with endoscopes for both manual and/or robotically operated configurations.

As the device moves through the anatomy, it may be configured in a highly tortuous

pathway, and it may be torqued or manipulated. It could be advantageous to configure the working channel sleeve apparatus such that the working channels descried herein are expandable and configured to prevent kinking, wrinkling, buckling, on either the inner diameter (i.d.) and/or outer diameter (o.d.). In some instances, the prevention of kinking, wrinkling and buckling could be advantageous to the passage of devices within the channels. The prevention of puncture through the wall is also important; the methods and apparatuses described herein, including the use of matched tools as described below, may be particularly helpful. In some examples these external working channels may be formed of a material that is both lubricious (e.g., slippery) and elastic (e.g., stretchy). This configuration may be advantageous for the easiest insertion, passage, sliding, and manipulation of a medical tool.

In some examples, a tool may be inserted directly through an external working channel (e.g., an expandable working channel) of a working channel sleeve.

In some examples, a liner insert tube (‘liner’) may be utilized in a working channel of a working channel sleeve. In some examples, the working channel(s) is/are expandable working channels that are configured to be used with a liner insert tube that maintains the patency of a channel or lumen through the external working channel. The liner insert tube may be coupled at a proximal end region of the device and to a distal end region of the device. The liner insert tube may be an enclosed tube or an open channel. The liner insert tube may be inserted into an external working channel of a working channel sleeve after an elongate medical device onto which the working channel sleeve is applied (e.g., worn, attached, etc.) has been positioned at or near a target region within the body.

The liner insert tube may be configured to be inserted into an external working channel to form a continuous and open inner channel through the elastic external working channel. The liner insert tube may be registered with the external working channel, e.g., using one or more wings, and/or engaging (e.g., locking) to the distal end region and/or proximal end region of the elongate medical device and/or tube. Precise attachment at the distal end enables precise movement of the tools that are used within the liner. The precise diameter of the liner tool assists in more precise movement of a tool through its lumen. The utilization of high-performance material or coatings on the liner enables precise movement of a tool through its lumen.

In general, the inner lumen or channel of the liner insert tube may include a lubricious surface (e.g., a surface having a low friction) to allow easy passage of a medical tool within, as well as easy of sliding of the liner within the expandable channel. In one example, the inner surface is hydrophilic. In other examples, the inner surface is a lower COF (coefficient of friction) plastic, including a fluoropolymer, or polyethylene, a polypropylene, or a plastic with a slip additive. The liner insert tube may also include engagement members (clips, locks, etc.) distally and/or proximally for securing to the elongate medical device and/or the tube. Thus, in some examples the liner insert tube may be configured to engage the distal end region of the elongate medical device so that it may secure (e.g., lock on to) the distal end region of the elongate medical device. In some examples the liner insert tube includes one or more projections (e.g., wings) at one or more positions along the length of the liner insert tube, or along the entire length of the liner insert tube. These projections may help keep the liner insert tube oriented relative to the elongate medical device.

The liner insert tube may be specifically designed as a matched set to the working channels, such that they work together with enhanced functionality. They may work together to generally form a channel or lumen that is held open along the length of the elongate medical device once positioned. A variety of different liner insert tubes may be used having different sizes/dimensions. For example, different liner insert tubes may have different diameters for passing different medical devices. Tools or ‘instruments’ may insert through the liner, including custom endoscopic tools and ‘standard’ endoscopic tools. Standard endoscopic tools are typically designed to fit through working channels of the following inner diameters: 4.2 mm, 3.8 mm, 3.2 mm, 2.8 mm, 2.0 mm. Multiple liner insert tubes may be inserted into different external working channels of the working channel sleeve apparatuses described herein. Since the working channels of the tube are normally maintained in a collapsed configuration but may be allowed to slide slightly with respect to the outer surface of the elongate medical device, the resulting structures may remain highly flexible while still having a relatively small outer profile when positioning within the body. Once positioned, the profile may be expanded by inserting one or more liner insert tubes and/or tools (e.g., directly, without requiring a liner insert tool).

The liner insert tube may be constructed using a variety of different techniques. For example, the liner insert tube may be an extrusion, typically of plastic or an elastomer. The liner insert tube may be a composite catheter shaft, including with a braid or with a fluoropolymer or ePTFE layer. In some examples the liner insert tube comprises a coil wound tube, or it may utilize a laser cut tube. The liner insert tube may be configured to have a key combination of parameters that are best achieved through the utilization of a composite structure: fantastic lubricity, good ‘pushability’ (e.g., high column strength and high axial stiffness,) low bend stiffness, good hoop stiffness, and a very reliably circular cross-section (even when it is bent through a tight radius of curvature).

In some examples the distal end of the liner insert tube may include a deflector. The deflector may be a blunt or rounded extension of the liner insert tube that may be configured to enable enhanced passage through the external working channel. The deflector may also be configured to prevent snagging as the liner insert tube is inserted into the external working channel. In some examples the deflector at the distal end of the liner insert tube is configured to have an atraumatic geometry so that it does cause anatomical damage as it emerges from the working channel. A deflectable tip portion may be useful for directing a tool as it exits the distal end region, thereby manipulation it in a particular direction (e.g., radially inward relative to the elongate medical tool). In some examples the deflector may include an eccentrically affixed pull wire.

For example, described herein are systems including tubes having one or more working channels configured to receive a medical tool inserted therethrough, each working channel may be positioned longitudinally along an exterior surface of the tube. The tube may be elastically expandable. Surprisingly, the working channel may expand without the use of elastic (i.e., it may effectively expand and contract, utilizing non-elastic materials. Both the tube and the one or more working channels may be comprised of a fabric material. The same material may be used for the body of the tube and the expandable (e.g., elastically or non-elastically expandable) working channels or different materials may be used.

In general, as used herein the term “expandable” may refer to elastically expandable so that the expandable structure (e.g., tube, external working channel, etc.) may be returned from an expanded configuration back to a collapsed configuration. In some examples the expandable tube may provide for ease of sliding by the use of a coating applied to the outside of the elastic component. For example, the coating could be a hydrophilic coating.

In some examples, the working channel sleeve apparatuses described herein may achieve the dual goals of high elasticity and low friction through the external working channels by using a composite material: e.g., a low friction material covering, encasing and/or coating an elastomeric core. In some examples the low-friction material is a material (e.g., yarn) that may cover or wrap around the elastomeric core. Typically, elastomers have high sliding friction. A low friction yarn could be, for example, a polyethylene, a polypropylene, or a fluoropolymer (for example, a PTFE). The composite material may be in a single layer or in multiple layers. For example, the composite material may include a knit structure of filament(s) (e.g., between 10-80 filaments, between 12-40 filaments, between 10-36 filaments, etc.) having a lubricious outer region over an elastic core.

The elasticity of the elastic material used may vary, in some cases within the same apparatus. For example, different elastic element materials may be used, and/or the filament count, filament thickness, etc. may be different among different elastic materials within the same or different apparatuses. In general, elastic materials (50%, 100%, 200%, 400%, 600%, 800%, 1000% stretch) tend to have high friction, including silicone, SPANDEX, and LYCRA. In general, plastics tend to be much less elastic (e.g., 2%, 4%, 6%, 8%, 10% stretch, referred to herein as “non-elastic”), and they tend to have dramatically lower friction. In some examples, a material such as spandex may be used. Spandex is a type of urethane that is a synthetic fiber known for its exceptional elasticity. Silicone may also be used, as it is highly biocompatible. Note that, as described in greater detail below, in some examples it may be particularly beneficial to provide non-elastic materials, in particular non-elastic filaments, for forming either or both the working channels and/or the core region of the apparatuses described herein.

In some examples the requisite elasticity could be achieved by a material that is both sufficiently elastic and sufficiently slippery.

However, in some examples, the requisite elasticity of the working channels (e.g., the overall structure) could be achieved by a structure that utilizes a material that is not elastic (“non-elastic”), but that can handle repeat deflections, including large magnitude deformations. Non-elastic materials may be “plastic” materials such as, but not limited to: PTFE, Polyester, UHMWPE, HDPE, and/or polypropylene. These materials are in contrast to traditional elastic materials such as (but not limited to): Spandex/Elastane/Latex and Silicone. For example, the non-elastic material forming the working channel(s) may have a Modulus of elasticity of greater than about 20,000 psi (e.g., 20,000 psi or greater, 30,000 psi or greater, 40,000 psi or greater, 60,000 psi or greater, 100,000 psi or greater, 200,000 psi or greater, 500,000 psi or greater, 750,000 psi or greater, etc.). The non-elastic material forming the working channel(s) may have relatively low coefficients of friction (e.g., less than about 0.5, 0.5 or less, 0.45 or less, 0.4 or less, 0.35 or less, 0.3 or less, 0.25 or less, 0.2 or less, 0.15 or less, 0.1 or less, etc.), particularly as compared to more elastic materials.

Alternatively or additionally, the working channel sleeve apparatuses described herein may achieve the dual goals of high elasticity and low friction through the working channel(s) by positioning or arranging a material having an elastomeric core of the filaments forming the working channel, so that it is spaced apart from (e.g. facing away from) materials with low sliding friction. For example, the elastomer “core” may not be encased, but it may be positioned in the cross section of the structure (the working channel) to ensure that devices moving within the working channel do not directly slide against the elastomeric material.

In examples in which the apparatus is knitted (e.g., the tube and/or elastic external working channels), the apparatus may be knitted on a computer-controlled knitting machine. Any appropriate needle gauge and/or stitch pattern may be used. For example, needle gages of knitting machine can be, for example, 10-14 or a 16-18 (with the 10 and 16 meaning needle gage and the 14 and 18 meaning the machine gage). A 16-18 machine enables much finer knitting.

The working channel sleeve apparatuses described herein may generally include a tube (e.g., a tubular body) that is configured to extend over an outer surface of an elongate medical device (e.g., catheter) and one or more external working channels formed along a length of the tube that are configured to receive a medical tool inserted therethrough. The one or more external working channels may also be referred to herein as a type of layflat tube. The elongate tubular body may also be referred to herein as an inner tube. The external working channels may be referred to equivalently herein as outer pockets. In some cases the working channels (e.g., outer pockets) are configured as external working channels that include an elastic element, e.g., may be formed of a knitted material that includes an elastic core, while in some examples the external working channels (e.g., outer pockets) do not include an elastic elements. Thus, the working channels described herein may be elastic or inelastic.

In working channel sleeve apparatuses including an inner tube and one or more outer working channels, the outer working channels may be formed of one or more filaments (or bundles of filaments) forming the outer working channels. The inner tube may also be formed of the same or a different filaments (or filaments or bundles of filaments) forming the inner tube, e.g., tubular body.

In particular, described herein are working channel sleeve apparatuses that are knitted. In some examples the working channel sleeve apparatus is formed by weft knitting. The working channel sleeve apparatus may be formed by warp knitting, including but not limited to tricot, Milanese knit, Raschel knit, and stitch-bonding. Any appropriately sized filament(s) may be used. For example in some examples the filament(s) forming the outer working channel(s) may be about 100 denier to 6000 denier for the outer wrap. Fiber(s) for the inner elastic core could be 400 to 6000 denier for the inner wrap. As used herein a filament or filaments (or bundles of filaments) forming all or part of a working channel sleeve apparatus may be referred to equivalently as a fiber or fibers (or bundle of fibers). These filaments may be natural or artificial and/or may be hybrid filaments as described herein.

The expandable tube of the working channel sleeve apparatus may be configured to slide over an elongate medical device. It may be unsecured between its distal and proximal ends. It may be secured at one or more points on the outer surface of the elongate medical device, such as the distal end region and/or the proximal end region. In some examples the tube may be secured at discrete points or intervals, between every 1-300 mm (e.g., between every 1-2 mm, 1-3 mm, 1-4 mm, 1-5 mm, 1-7 mm, 1-10 mm, 1-15 mm, 1-20 mm, 1-25 mm, 5-10 mm, 5-15 mm, 5-20 mm, 5-25 mm, 5-30 mm, 5-35 mm, 5-40 mm, 10-20 mm, 20-30 mm, 10-100 mm, 10-200 mm, 100 mm-200 mm, 100 mm-300 mm, etc.). The expandable tube may be attached circumferentially or at one or more points or lines. The expandable tube may be attached locally and continuously. The expandable tube may be configured to slide relative to the outer surface of the elongate medical device in regions where it is not attached, which may help prevent wrinkling and/or blocking of the external working channel(s). The expandable tube may be configured to have a frictional adhesion relative to the outer surface of the elongate medical device in regions where it is not attached, which may prevent it from moving and wrinkling and bunching. Wrinkling may occur when the material forming he external working channel bunches up and in some cases folds over itself, which may reduce the effective size of the channel and/or may cause jamming of a tool (including liner tool) inserted into the channel.

In some examples, the working channels can be created as a sewn structure from flat

cloth. The cloth can be an expandable cloth, such as a stretchy mesh. A lubricious coating can be used with the cloth (e.g., mesh) so that tools can readily slide within the working channel. The cloth (e.g., mesh) may have openings or pores. The pores may change shape as the cloth is stretched or compressed, including expanding and contracting and distorting from their original shape. The material could be polyester or polypropylene or Teflon with an elastic stretch core that could be, for example, urethane (including spandex), or silicone. In general, the pore size of the working channels (and/or inner tubular body) can vary from about 0.05 mm to about 4 mm, in knitted, woven and/or cloth embodiments.

As mentioned, any of these working channel sleeve apparatuses may include or be used with a liner, including but not limited to a liner insert tube. In any of the apparatuses described herein a liner may be used within the external working channels. The material forming the working channel may be stretchy enough to hold and control the liner. If a cloth material forming the working channel is too stretchy, the liner may distort the material, and may poke out from the working channel, and/or may buckle or wrinkle as the apparatus is put through curvature. If the cloth material forming the working channel is not stretchy enough, the liner insertion force may become too high, making it difficult, if not impossible, to insert, and it may stall out in a wrinkle as it tries to advance around a corner or a tortuous path. The performance of the material, e.g., knit material and/or fabric material, may be modulated by changing the orientation of the weft and the warp. For example, a woven cloth can be sewn in-line, or can be sewn at a 45 degree bias. In some examples the cloth can be comprised of yarns with filament counts. Filament counts can very, for example, from 5 to 100. High filament count yarns can create more lubricious cloths.

In any of these examples of working channel sleeve apparatuses, the working channels can be created as a laminated structure. For example, by inserting a layer of a thermoplastic elastomer between layers of cloth, the working channels can be adhered in a manner that is non-penetrating and elastic. The bonding may occur with the assistance of heat, pressure, and the passage of time. Similarly, structures can be laminated with other material layers. For example, elastomeric layers can be laminated with fiber layers, with layers that are selective cut-out (for example, by lasers, die-cutting, or CNC knife-cutting profiles).

Any of the working channel sleeve apparatuses described herein may be used with an elongate medical device. For example the elongate medical device may include a catheter, endoscope, overtube, etc. In some examples the elongate medical device is a rigidizing device (e.g., a rigidizing system). Although examples including rigidizing (e.g., selectively rigidizing member) are provided herein and incorporated by reference in their entirety, including but not limited to those described in international patent application PCT/US2019/042650 (“DYNAMICALLY RIGIDIZING COMPOSITE MEDICAL STRUCTURES”, filed Jul. 19, 2019, herein incorporated by reference in its entirety), the systems and methods described herein are not limited to rigidizing apparatuses, or any particular type of rigidizing apparatus.

The working channels described herein may be elastically expandable and may be configured to contour the shape of the medical tool or a liner insert tube inserted therethrough. As mentioned, in some examples it may be advantageous to include a liner insert tube that may be inserted into the working channel, and particularly expandable working channels, in order to maintain patency of the working channel for insertion of one or more medical tools. The liner insert tube described herein may include a closed or open lumen (e.g., open along all or some of the elongate length of the liner insertion tube). The liner insert tube may also be equivalently referred to herein as a liner insert or liner insert channel. In any of these examples the liner insert tube may include a lubricous inner lumen that extends therethrough. The outer surface of the liner insert tube may be lubricious and/or may include a lubricous coating or material. In some examples the inner lumen may be hydrophilic or hydrophobic (e.g., may include a hydrophilic or hydrophobic coating) on the inner and/or outer surface of the liner insert tube. The liner insert tube may be used when inserting a tool into an external working channel, particularly when it would be beneficial to expand or hold the expandable working channel open for inserting a tool, particularly a tool that may otherwise catch or snag on the external working channel (e.g., when the external working channel is formed of a knit, woven or braided material, or any other material having pores/openings).

The liner insert tube may be inserted into one of the one or more working channels from a proximal end to a distal end of the working channel. Once inserted, a medical tool may be passed through the lumen of the liner insert tube for use at or near the distal end of the elongate medical device.

As mentioned above, the external working channels may generally be constructed of one or more filaments (e.g., knit, woven, etc.) having a low friction outer region that at least partially encloses an elastic inner region. In any of these apparatuses the external working channels may also include one more filaments that do not include an elastic core (and/or may be non-elastic). For example, the external working channels may be a mix of elastic and non-elastic (or less elastic) filaments.

The body of the tube and/or the working channels of the working channel sleeve apparatus may be formed as a non-uniform weave, braid or knit pattern. In some examples, the weave pattern of the tube body and the one or more external working channels is the same. In some examples, the weave pattern of the one or more external working channels is different than a weave pattern of the body of the tube. Additionally, the tube can comprise more than one segment defined by a change in a weave pattern of the (e.g., along the length of the tube). Similarly, the one or more working channels can have more than one segment defined by a change in the weave pattern of the fabric, which may allow modulation of the elasticity of the length of the tube and/or may bias a tool or the linear insert tube to be retained at a particular longitudinal position of the tube.

As mentioned, the tube body of the working channel sleeve apparatus may be formed of a woven braid of one or more filaments. The one or more filaments may be formed of a core of an elastic material configured to elastically expand and retract and a lubricious coating, winding, wrapping and/or layer over the elastic core. In some examples the lubricious coating/winding/wrapping/layer may be a wrap substantially or completely encompassing the elastic core, forming a coil-wound filament.

The tube may include any appropriate number of external working channels, such as between 1 and 12 (e.g., between 1-10, between 1-9, between 1-8, between 1-7, between 1-6, between 1-5, between 1-4, between 1-3, between 1-2, or just one). The channels could be the same shape or size, or they may be different shapes or sizes.

The tube body of the working channel sleeve apparatus may generally conform to the shape of the elongate medical device as it is bent or otherwise navigated through the body. Apparatuses including the tube comprising the external working channels may be configured to allow bending without substantially increasing the stiffness of the elongate medical device or without. Each of the external working channels of the working channel sleeve apparatus may be expandable to accommodate a tool and/or a liner insert tube inserted therethrough. The channels may expand independent of one another and independent of the expandable tube. Channel expansion of one channel may have a relationship to the potential channel expansion of another, as the structures are co-joined. Each of the channels may be positioned around a perimeter of the expandable tube. In some examples, when the working channel sleeve has multiple external working channels, the external working channels can be equidistant from one another on the exterior surface of the tube body of the working channel sleeve apparatus. For example, where there are four channels, each of the channels may be separated by 90 degrees on center about a longitudinal axis of the expandable tube. The tube and the working channels of the working channel sleeve apparatus may include or be composed of materials that are elastomeric, plastic, and/or fabric. The channels may have spaces between them, or they may be immediately adjacent to each other. In some examples the channels may overlap. The channels may be attached over their entire length. The channels may be attached at discrete points or lines. The channels may be attached at an intermediate value between a point or line and over their entire length.

Any of the elongate medical devices described (e.g., catheters, endoscopes, etc.) may include an imaging element positioned at a distal end, such that the imaging element may be used to identify and assist in operation of tools passed through the external working channels of the working channel sleeve apparatus during a medical procedure. The elongate medical device can also have corresponding imaging element positioned at a proximal end.

For example, described herein are working channel sleeve apparatuses including an expandable external working channel, the system comprising: an elongate medical device; a tube extending over an outer surface of the elongate medical device; and one or more external working channels formed along a length of the tube and configured to receive a medical tool inserted therethrough. Thus a working channel sleeve apparatus may also include the elongate medical device onto which the elongate body of the working channel sleeve is worn or applied, forming the elongate external channels extending down the length of the elongate medical device.

In any of these apparatuses, the tube may comprise a woven, knit or braided tube, or a combination thereof. In some examples the tube is formed of one or more elastic and lubricious filaments. The tube may be slidably connected to the elongate medical device. The tube may be coupled to a proximal end region and to a distal end region of the elongate medical device.

Any of the apparatuses (e.g., devices, systems, etc.) described herein may be formed of a material (or materials) that is/are laminated.

In any of these systems, the elongate medical device may comprise a catheter, overtube or an endoscope. In some examples, the elongate medical device comprises a selectively rigidizing device.

Any of these systems may include a liner insert tube configured to be inserted within one of the one or more expandible working channels and removably coupled to a distal end of one or both of the elongate medical device and tube. The liner insert tube may have a hydrophilic interior and or exterior surface. In some examples the liner insert tube comprises one or more wings extending proud of a side of the liner insert tube configured to limit torque of the liner insert tube within the one of the one or more expandible working channels. The wings can provide distal to axial registration, and may prevent torquing and lateral motion. In some examples the liner insert tube may include a deflector at a distal end opening of the liner insert tube configured to deflect a tool exiting the liner insert tube away from a radially outward direction relative to the elongate medical device.

The inner tube of the working channel sleeve apparatus may be formed of one or more filaments, including in particular filaments comprising an inner elastic material and an outer lubricious material. The tube may be formed of a coil-wound filament comprising an elastic core wrapped in a lubricious material. In this instance, the combined entity has important resultant properties: the elasticity of the elastomer and the surface-contacting lubricity of the lubricious material. In some examples, the lubricious material comprises a polypropylene, a polyethylene, or a polytetrafluoroethylene. The tube may have a non-uniform weave pattern, or a uniform weave pattern. In some examples, the tube may be sheet of elastomer (e.g., elastomeric material) with cutouts so that it resembles a fine mesh potentially working for this application.

For example, a working channel sleeve system including an expandable external working channel may include: an elongate medical device having a flexible or selectively rigidizable body; a knit or woven tube extending over an outer surface of the elongate medical device wherein the tube is coupled to a distal end region and a proximal end region of the elongate medical device and is slidable relative to the outer surface over at least a portion of the flexible or selectively rigidizable body between the distal end region and the proximal end region; and one or more external working channels formed along a length of the tube and configured to receive a medical tool inserted therethrough.

In some examples the system (e.g., the working channel sleeve system including an expandable external working channel) includes: an elongate medical device; a knit or woven tube extending over an outer surface of the elongate medical device, the tube comprising one or more external working channels extending along a length of the tube and configured to receive a medical tool inserted therethrough; and a liner insert tube configured to be inserted within one of the one or more expandible working channels and removably coupled to a distal end of one or both of the elongate medical device and tube.

Also described herein are methods of using any of these working channel sleeve apparatuses (e.g., systems, devices, etc.). For example, a method of positioning a tool within a body may include: inserting an elongate medical device into the body in a flexible configuration; inserting a liner insert tube into an expandible working channel of a knit or woven tube extending over an outer surface of the elongate medical device, so that the external working channel expands to accommodate the liner insert tube; and inserting a working tool through the liner insert tube and out of a distal end of the liner insert tube.

Any of these methods may in include performing a medical procedure in the body with the working tool.

In some examples, the method may include locking the distal end of the liner insert tube at a distal end region of the elongate medical device. Alternatively or additionally, the liner insert tube may be locked at the proximal end of the elongate medical device. The length of the liner insert tube may be allowed to slide relative to the inner surface of the working channel as the elongate medical device is navigated within the body, while the liner insert tube is fixed at the distal (and/or proximal) end of the elongate medical device.

Any of these methods may include maintaining patency of the liner insert tube while it is inserted into the expandible working channel.

The methods described herein may be used with a rigidizing elongate medical device. For example, any of these methods may include rigidizing the elongate medical device. In some examples the method may include rigidizing the elongate medical device before inserting the liner insert tube. Any of these methods may include deflecting the working tool radially inwards as it is extended out of the distal end of the liner insert tube using a deflector at a distal end region of the liner insert tube. Inserting the liner insert tube into the expandible working channel may include engaging one or more wings on the liner insert tube with the expandible working channel. In some examples inserting the liner insert tube comprises sliding the liner insert tube against a lubricious outer surface of one or more filaments forming the expandible working channel of the knit or woven tube.

As mentioned above, the expandible working channel of the knit or woven tube may be formed of one or more filaments comprising an inner elastic material and an outer lubricious material. Inserting the liner insert tube may expand the expandible working channel from a collapsed configuration in which the expandible working channel is flush against the outer surface of the elongate medical device.

For example, a method of positioning a tool within a body may include: inserting an elongate medical device into a body in a flexible configuration, so that a knit or woven tube extending over an outer surface of the elongate medical device may slide relative to the outer surface; positioning a distal end of the elongate medical device near a target region of the body; inserting a liner insert tube into an expandible working channel of the tube, so that the external working channel expands to accommodate the liner insert tube; and locking a distal end of the liner insert tube to a distal end of the elongate medical device and/or the tube, wherein the liner insert tube maintains patency of a lumen extending through the liner insert tube.

A method of positioning a tool within a body may include: inserting an elongate medical device comprising a rigidizing member into a body while the rigidizing member is in a flexible configuration, so that a knit or woven tube extending over an outer surface of the elongate medical device may slide relative to the outer surface; positioning a distal end of the elongate medical device near a target region of the body; rigidizing the elongate medical device; inserting a liner insert tube into an expandible working channel of the tube, so that the external working channel expands to accommodate the liner insert tube; and inserting a working tool through the liner insert tube and out of a distal end of the liner insert tube. Alternatively, rigidization could occur at different steps in the process.

The methods and apparatuses described may be used with and/or may modify any of the methods and apparatuses described in International Patent Application No. PCT/US2016/050290, filed on Sep. 2, 2016, titled “DEVICE FOR ENDOSCOPIC ADVANCEMENT THROUGH THE SMALL INTESTINE,” published as WO 2017/041052, International Patent Application No. PCT/US2018/042946, filed on Jul. 19, 2018, titled “DYNAMICALLY RIGIDIZING OVERTUBE,” published as WO 2019/018682, International Patent Application No. PCT/US2019/042650, filed on Jul. 19, 2019, titled “DYNAMICALLY RIGIDIZING COMPOSITE MEDICAL STRUCTURES,” published as WO 2020/018934, International Patent Application No. PCT/US2020/013937 filed on Jan. 16, 2020, titled “DYNAMICALLY RIGIDIZING COMPOSITE MEDICAL STRUCTURES,” and PCT/US2021/034292, filed on May 26, 2021, entitled “RIGIDIZING DEVICES” the entireties of which are incorporated by reference herein.

For example, described herein are external working channel assemblies. These assemblies may include: a core region configured to extend over an outer surface of an elongate, flexible member; and an external working channel extending along an outer surface of the core region, wherein the external working channel is formed of one or more non-elastic filaments that are configured to slide over each other to expand the external working channel to accommodate a tool inserted through the external working channel. In some examples the core region and the external working channels may be collectively referred to as the working channel assembly.

In some examples an external working channel assembly, the assembly comprising: a core region configured to extend over an outer surface of an elongate, flexible member; and an external working channel extending along an outer surface of the core region, wherein the external working channel is formed of one or more non-elastic filaments forming a plurality of pore openings, wherein the one or more non-elastic filaments are configured to slide over each other to change dimensions of pore openings of the plurality of pore openings to expand the external working channel and accommodate a tool inserted through the external working channel.

In some cases the working channel assembly may also include the elongate flexible member onto which the core region and external working channels are attached. For example, described herein an external working channel assembly includes: an elongate, flexible member; a core region extending over an outer surface of the elongate, flexible member, wherein the core region is coupled to a proximal end region of the elongate, flexible member and to a distal end region of the elongate, flexible member; and an external working channel extending along an outer surface of the core region, wherein the external working channel is formed of one or more non-elastic filaments that are configured to slide over each other to expand the external working channel to accommodate a tool inserted through the external working channel.

The external working channel may include a plurality of pore openings formed by the one or more non-elastic filaments and configured to change dimension as the one or more non-elastic filaments slide over each other. The external working channel may be knitted or braided. The one or more non-elastic filaments comprise a plastic material having a modulus of elasticity of greater than 50,000 psi. For example, the one or more non-elastic filaments may comprise one of: polytetrafluoroethylene (PTFE), polyester, ultra-high-molecular-weight polyethylene (UHMWPE), high density polyethylene (HDPE), and polypropylene. The one or more non-elastic filaments may include a plastic material having a coefficient of friction of 0.5 or less. The core region may be formed of one or more filaments. The core region may be formed of at least some of the filaments of the one or more filaments forming the external working channel.

Any of these apparatuses (e.g., assemblies) may include a plurality of external working channels extending along the outer surface of the core region. In any of these apparatuses, the external working channel may be configured to have an anisotropic stretch profile with a lower hoop stretch than an axial stretch.

As mentioned above, in any of these apparatuses, the core region may be coupled to the outer surface of the elongate flexible member at a distal end region or the elongate flexible member and a proximal end region of the elongate flexible member, but is not secured to the elongate flexible member between the distal end region and the proximal end region. The elongate flexible member may include an endoscope or an overtube. The elongate flexible member may include a rigidizing device. The core region and the external working channel may include a multi-lumen braid. The core region and the external working channel may be formed as a horizontal knit.

In general, also described herein are tools, including tools forming part of the assembly. In some examples, the apparatuses described herein include a matched pair of tools configured to be inserted together through the external working channel, wherein a first tool of the pair of tools comprises a steerable distal end configured to steer a second tool of the pair of distal tools as it exits the external working channel.

Any of these apparatuses, and in particular the working channels, may include a coating (e.g., lubricious coating). For example, an external working channel may include a lubricious coating.

Any of these apparatuses may include a deflector at a distal end of the external working channel configured to defect a tool extending from a distal end of the external working channel radially away from the distal end of the elongate, flexible member.

Also described herein are apparatuses (e.g., external working channel assemblies) that are configured so that the core region and external working channels do not significantly reduce flexibility of the medical device, e.g., elongate flexible member, onto which they are attached, but that prevent binding or bunching-up of the core region and external working channel(s) by controlling the coefficient of friction between the core region and the outer surface of the elongate flexible member. For example, described herein are external working channel assemblies comprising: an elongate, flexible member; a core region extending over an outer surface of an elongate, flexible member, wherein the core region is coupled to the outer surface of the elongate flexible member at a distal end region or the elongate flexible member and a proximal end region of the elongate flexible member, but is not secured to the elongate flexible member between the distal end region and the proximal end region; and an external working channel extending along an outer surface of the core region, wherein the external working channel is configured to accommodate a tool inserted through the external working channel, further wherein a coefficient of friction between core region and the outer surface of the elongate flexible member is between 0.3 and 1 to prevent bunching of the core region and external working channel when operating the assembly.

The coefficient of friction may be, e.g., between about 0.35 and 1, between about 0.4 and 1, between about 0.45 and 1, between about 0.5 and 1, etc.

In general, the external working channel may be formed of one or more non-elastic filaments. The external working channel may comprise a plurality of pore openings formed by the one or more non-elastic filaments and configured to change dimension as the one or more non-elastic filaments slide over each other. The external working channel may be knitted or braided. The one or more non-elastic filaments may comprise a plastic material having a modulus of elasticity of greater than 50,000 psi. For example, the one or more non-elastic filaments may comprise one of: polytetrafluoroethylene (PTFE), polyester, ultra-high-molecular-weight polyethylene (UHMWPE), high density polyethylene (HDPE), and polypropylene. The one or more non-elastic filaments may comprise a plastic material having a coefficient of friction of 0.5 or less. The core region may be formed of one or more filaments, including any of these filaments described above. The core region may be formed of at least some of the filaments of the one or more filaments forming the external working channel.

Any of these apparatuses may include a plurality of external working channels extending along the outer surface of the core region. The external working channel may be configured to have an anisotropic stretch profile with a lower hoop stretch than an axial stretch. The elongate, flexible member may include an endoscope or an overtube. The elongate, flexible member may comprise a rigidizing device. The core region and the external working channel may comprise a multi-lumen braid. The core region and the external working channel may comprise a horizontal knit.

As mentioned, any of these apparatuses may include a matched pair of tools configured to be inserted together through the external working channel, wherein a first tool of the pair of tools comprises a steerable distal end configured to steer a second tool of the pair of distal tools as it exits the external working channel. Alternatively or additionally, any of these apparatuses may include a deflector at a distal end of the external working channel(s) configured to defect a tool extending from a distal end of the external working channel radially away from the distal end of the elongate, flexible member.

Also described herein are apparatuses (e.g., systems, devices, assemblies, etc.), that include an expandable external working channel. For example, a system may include: an elongate medical device; a core tube extending over an outer surface of the elongate medical device; and one or more expandable working channels formed along a length of the tube and configured to receive a medical tool inserted therethrough, wherein the one or more expandable working channels is formed of one or more filaments, each of the one or more filaments comprising an inner elastic material and an outer lubricious material. As mentioned above, in some examples the tube is formed of a coil-wound filament comprising an elastic core wrapped in a lubricious material. The lubricious material may comprise a polypropylene, a polyethylene, or a polytetrafluoroethylene.

As mentioned above, also described herein are methods of making any using any of the apparatuses (e.g. assemblies) described herein. For example, a method of positioning a tool within a body, the method comprising: inserting an elongate medical device into the body in a flexible configuration; inserting a liner insert tube into an expandible working channel of a tube extending over an outer surface of the elongate medical device, so that the expandable working channel expands to accommodate the liner insert tube; steering a distal end of the liner insert tube; and inserting a working tool through the liner insert tube and out of a distal end of the liner insert tube, wherein the working tool is steered as it is extended of the distal end of the liner insert tube. The methods described herein may include performing a medical procedure in the body with the working tool. The methods described herein may include locking the distal end of the liner insert tube at a distal end region of the elongate medical device.

In any of these methods, the elongate medical device may be configured to allow at least a portion of a length of the tube to slide relative to the outer surface of the elongate medical device as the elongate medical device is navigated within the body, and/or to limit the sliding (e.g., by controlling the coefficient of friction between the core region and the outer surface of the elongate medical device).

Any of these methods may include maintaining patency of the liner insert tube while

it is inserted into the expandible working channel. Any of these methods may include rigidizing the elongate medical device. For example, the method may include rigidizing the elongate medical device before inserting the liner insert tube.

In some examples the method may include deflecting the working tool radially inwards as it is extended out of the distal end of the liner insert tube using a deflector at a distal end region of the liner insert tube. Inserting the liner insert tube into the expandible working channel may include engaging one or more wings on the liner insert tube with the expandible working channel. Inserting the liner insert tube may comprise sliding the liner insert tube against a lubricious outer surface of one or more filaments forming the expandible working channel of the knit or woven tube.

As mentioned above, the expandable working channel of the knit or woven tube may be formed of one or more non-elastic filaments. The expandible working channel of the knit or woven tube may be formed of one or more filaments comprising an inner elastic material and an outer lubricious material. In any of these methods, inserting the liner insert tube may expand the expandible working channel from a collapsed configuration in which the expandible working channel is flush against the outer surface of the elongate medical device.

Also described herein are systems including an expandable external working channel, the system comprising: an elongate medical device having a flexible or selectively rigidizable body; a knit, woven or braided tube extending over an outer surface of the elongate medical device and formed of one or more non-elastic filaments; and one or more knit, woven or braided external and expandable working channels integrally formed along a length of the tube and configured to receive a medical tool inserted therethrough, wherein the knit, woven or braided external and expandable working channels are formed of one or more non-elastic filaments.

Also described herein are methods of positioning a tool within a body, the method comprising: inserting an elongate, flexible member into a body so that an external working channel coupled to a core region extending over an outer surface of the elongate, wherein the external working channel is formed of one or more non-elastic filaments that are configured to slide over each other; positioning a distal end of the elongate, flexible member near a target region of the body; inserting a tool or a liner insert tube into the external working channel expands to accommodate the tool or liner insert tube by sliding the non-elastic filament relative to each other. Any of these methods may include: locking a distal end of the liner insert tube to a distal end of the elongate flexible member and/or the core region, wherein the liner insert tube maintains patency of a lumen extending through the liner insert tube.

For example, a method of positioning a tool within a body may include: inserting an elongate medical device comprising a rigidizing member into a body while the rigidizing member is in a flexible configuration, so that a knit, braided or woven tube extending over an outer surface of the elongate medical device may slide relative to the outer surface; positioning a distal end of the elongate medical device near a target region of the body; rigidizing the elongate medical device; inserting a liner insert tube into an expandible working channel of the tube, so that the expandable working channel expands to accommodate the liner insert tube; and inserting a working tool through the liner insert tube and out of a distal end of the liner insert tube.

All of the methods and apparatuses described herein, in any combination, are herein contemplated and can be used to achieve the benefits as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the methods and apparatuses described herein will be obtained by reference to the following detailed description that sets forth illustrative embodiments, and the accompanying drawings of which:

FIG. 1 shows an example of a system including an external working channel and an elongate medical device.

FIG. 2 shows an example of a system including an external working channel system in combination with an elongate medical device (e.g., a rigidizing device) and a liner insert tube.

FIGS. 3A and 3B illustrate schematic cross-sectional views of a tube including four expandable external working channels around the perimeter of the tube.

FIG. 4 schematically illustrates the expansion of four external working channels around the exterior of a tube.

FIGS. 5A and 5B show examples of a proximal end of a tube having an external

channel over an elongate medical device, showing the insertion of a liner insert tube from the proximal end of the external working channel.

FIG. 6 shows an example of an elongate medical device, in this example a rigidizing device.

FIG. 7 schematically illustrates an example of a hybrid or composite fiber comprising an elastic core wrapped in a lubricious material.

FIGS. 8A and 8B schematically illustrate segments of a rigidizing tube showing examples of contouring between the external working channels and the exterior of the rigidizing tube.

FIGS. 9A-9E show schematic illustrations of one example of an elongate medical

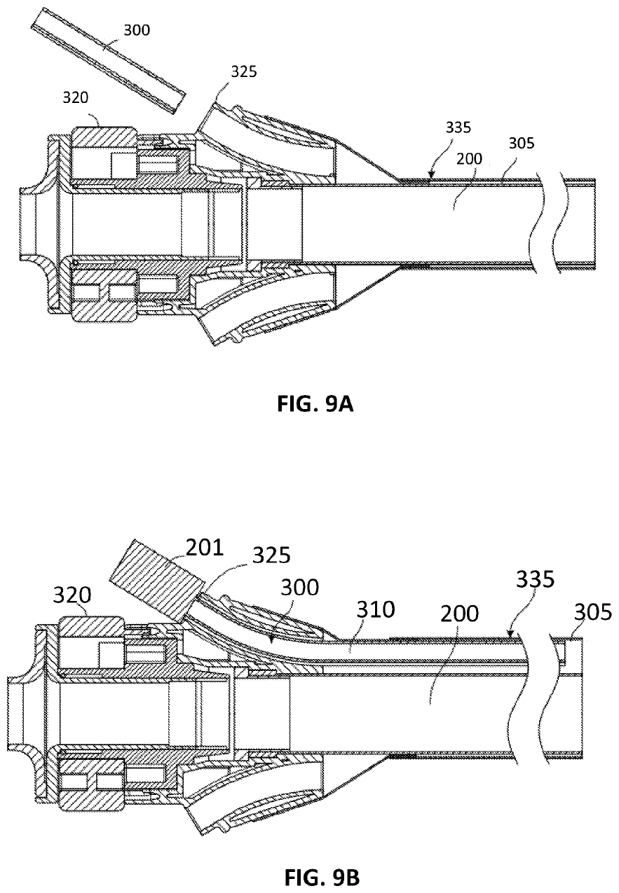

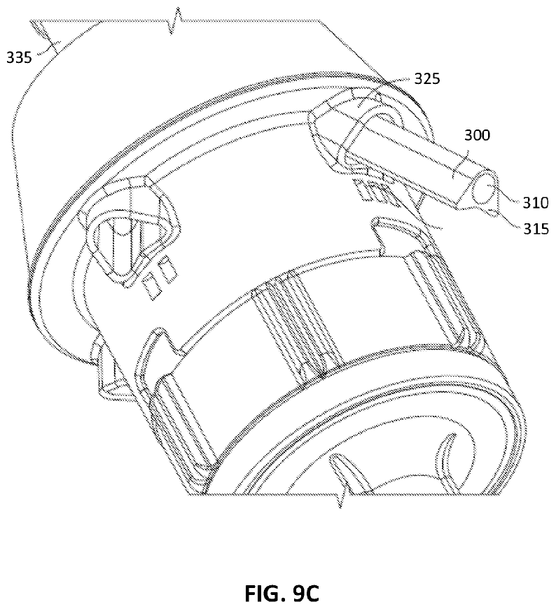

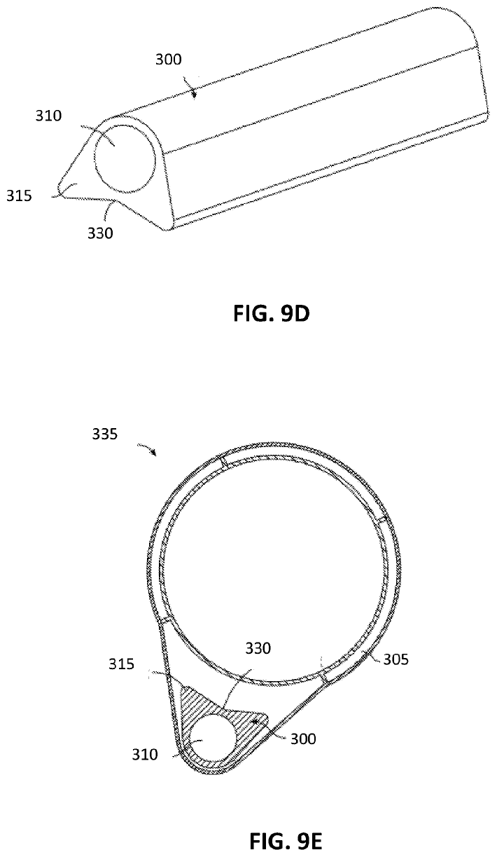

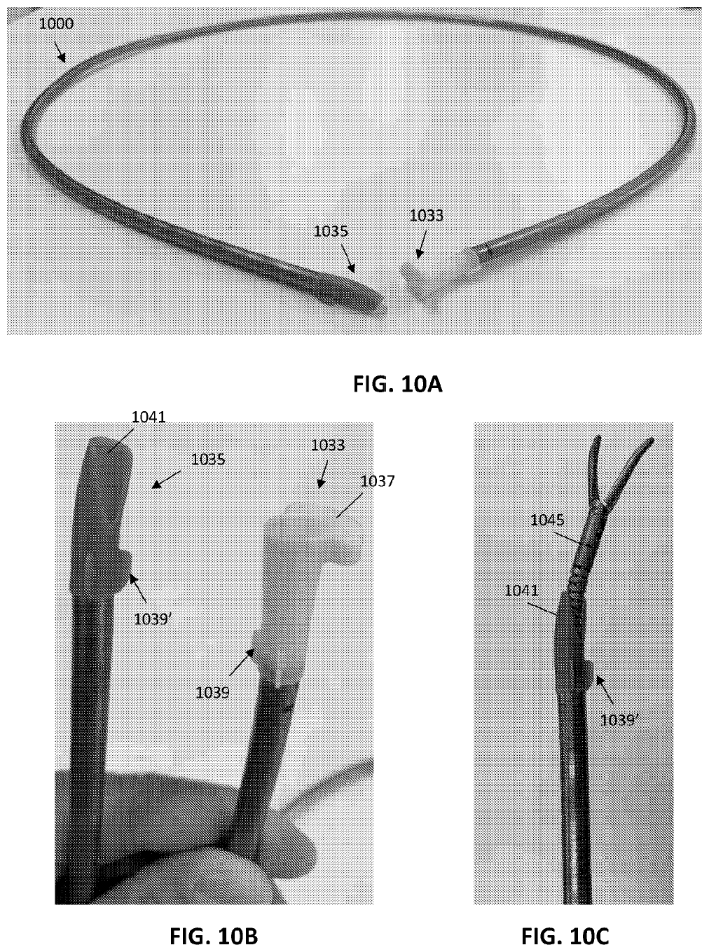

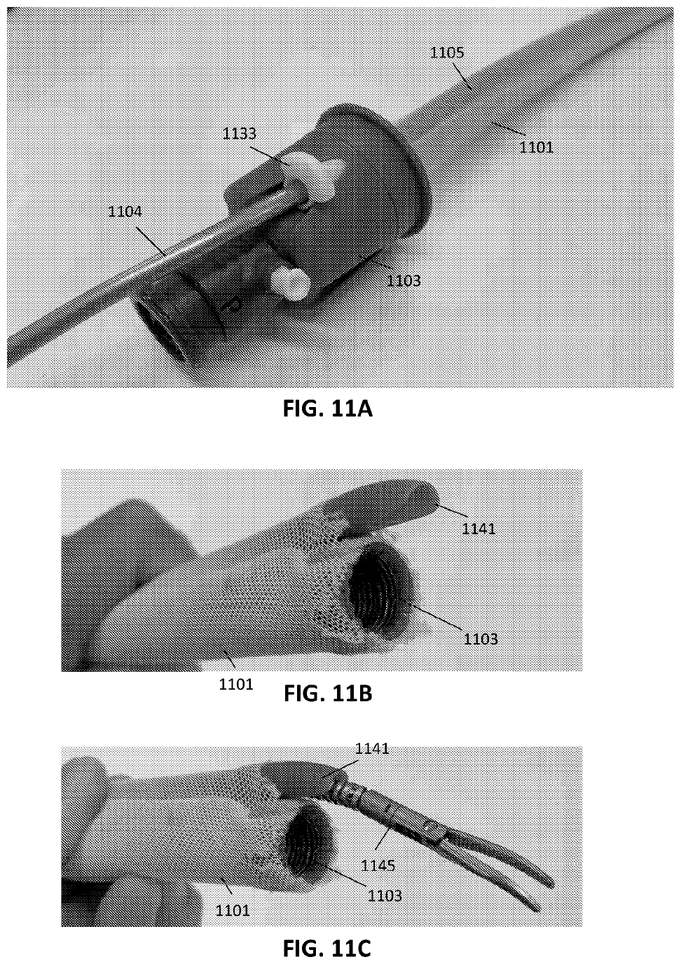

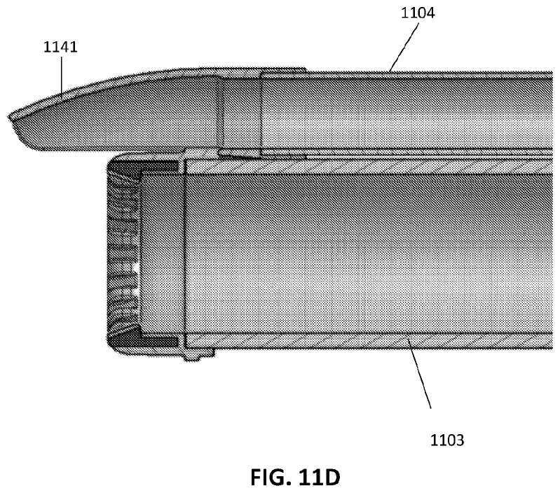

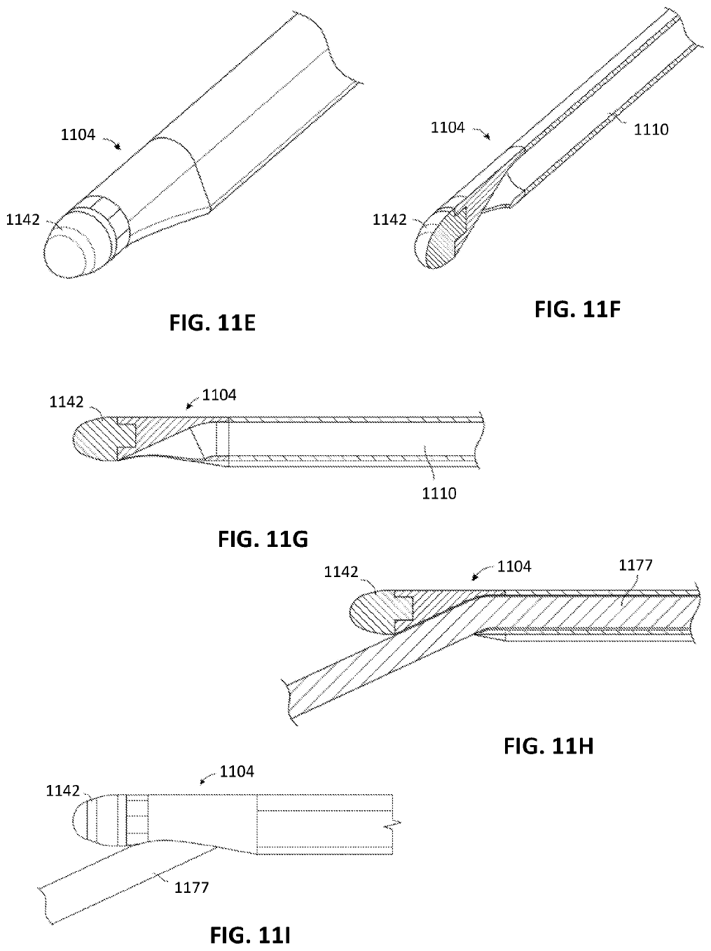

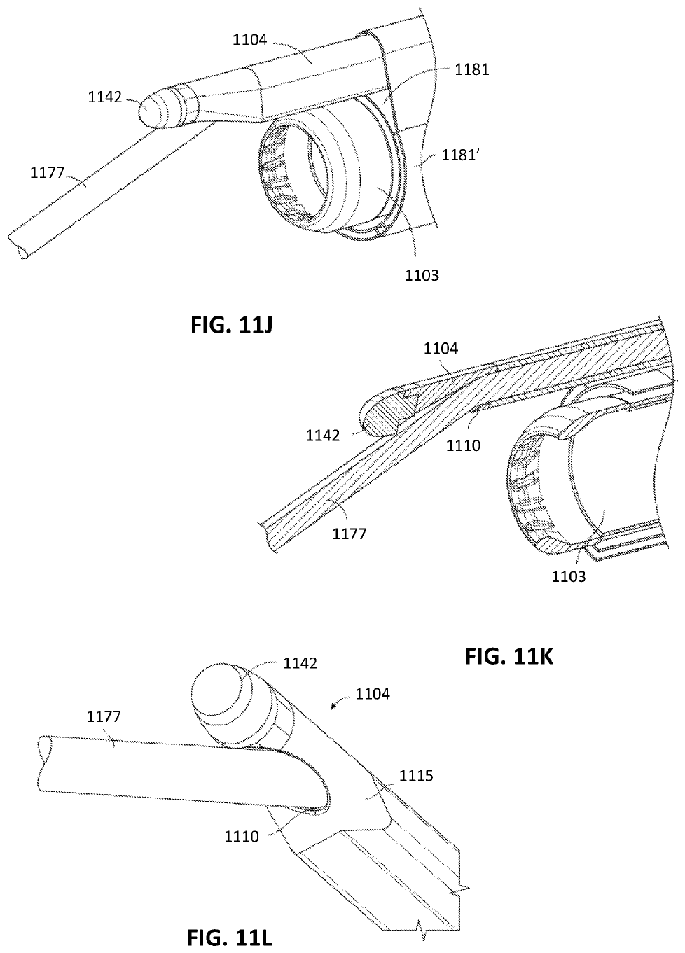

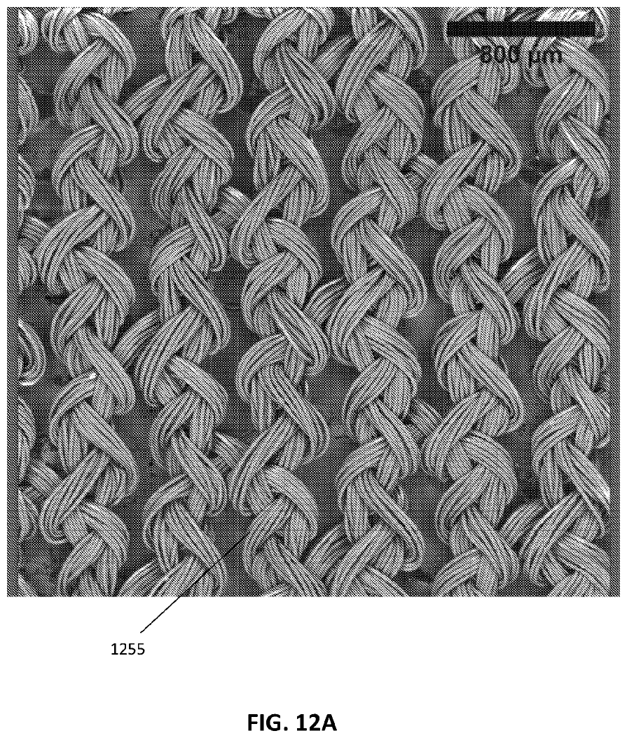

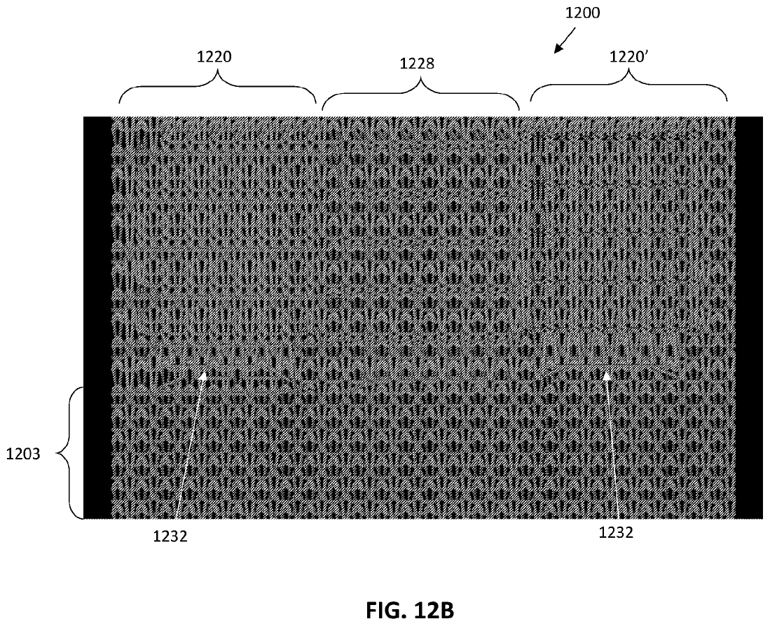

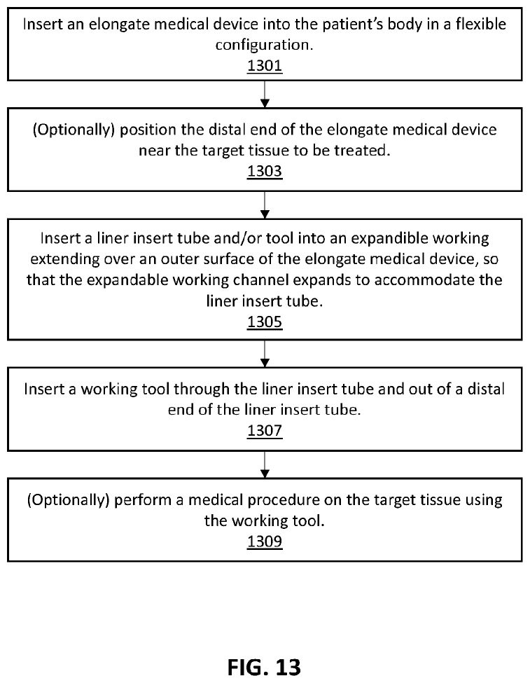

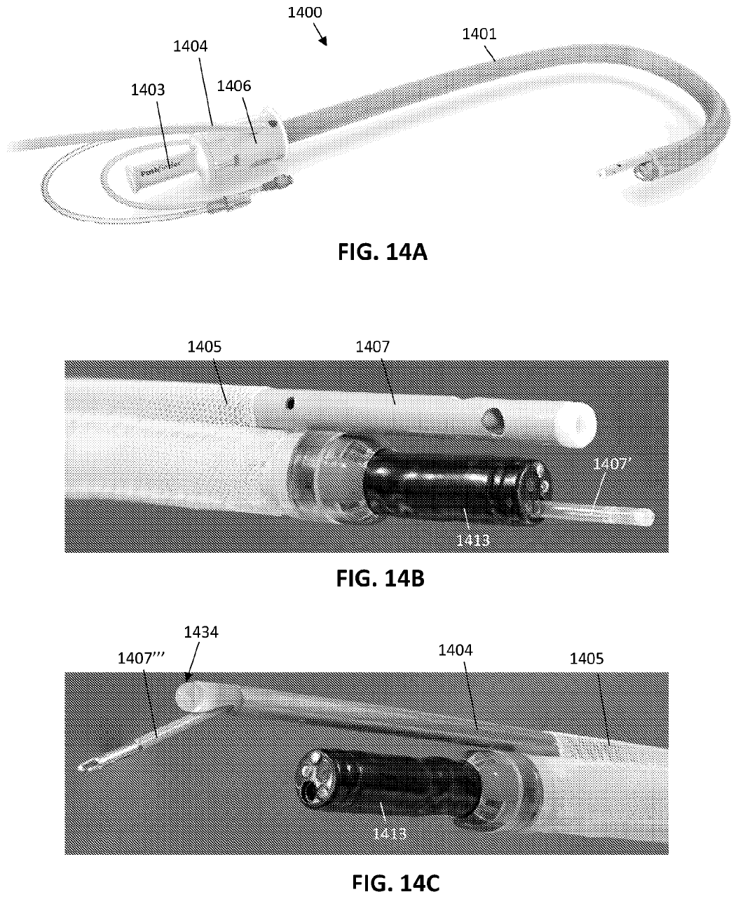

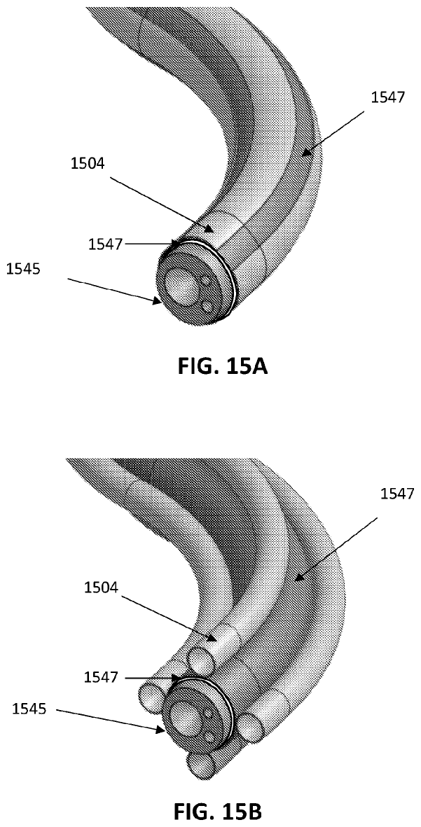

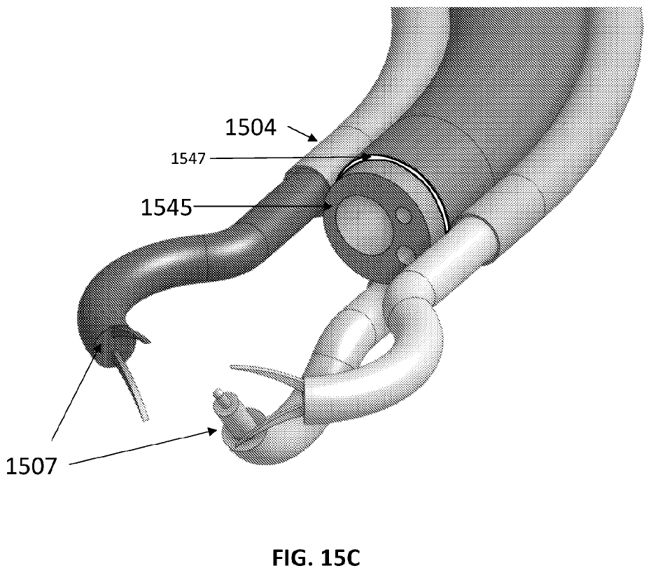

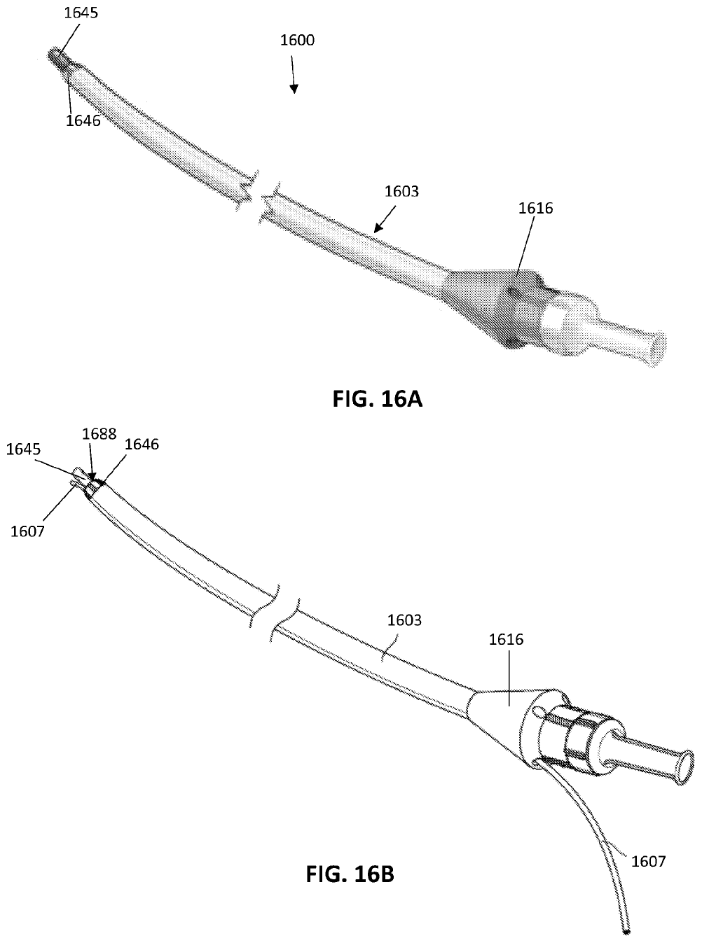

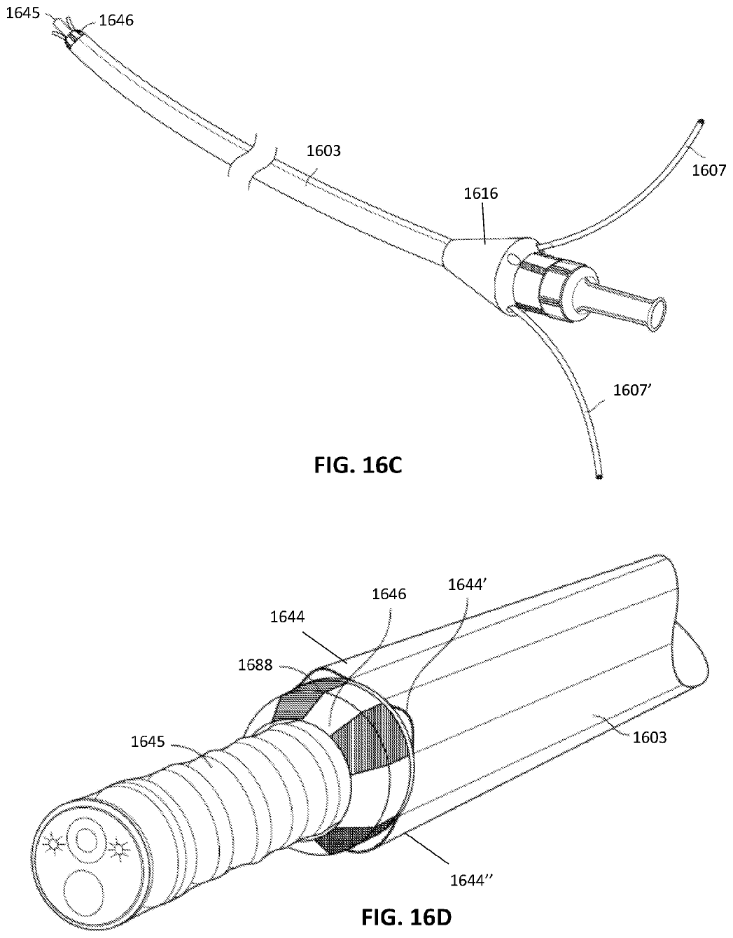

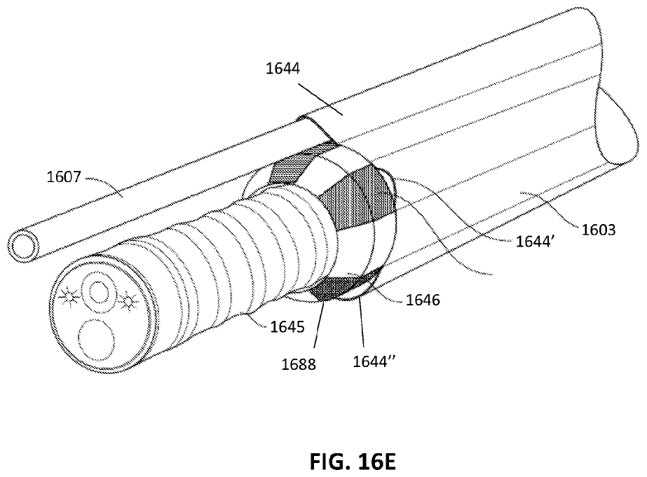

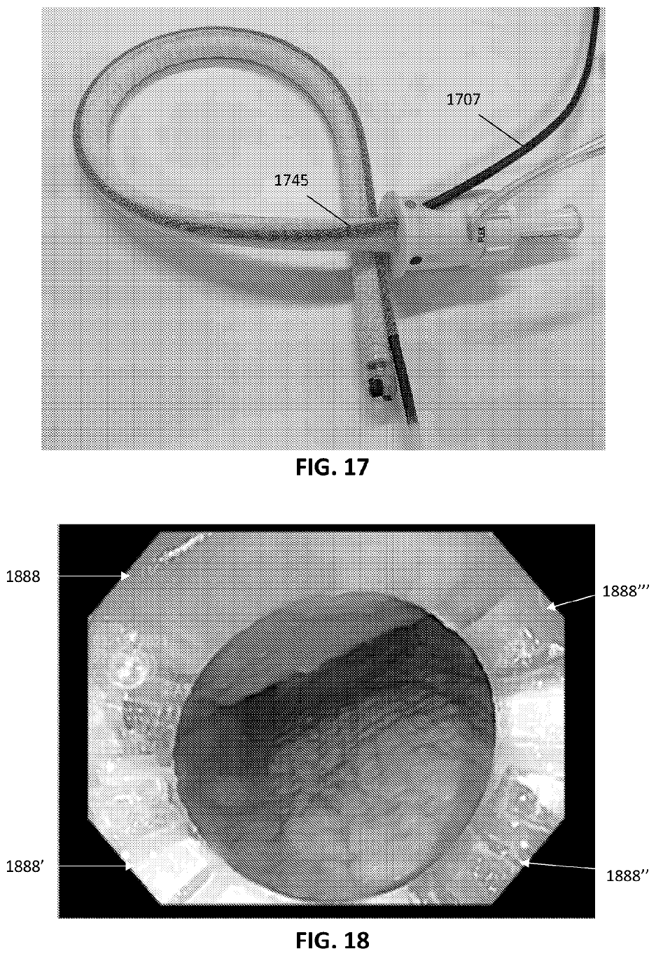

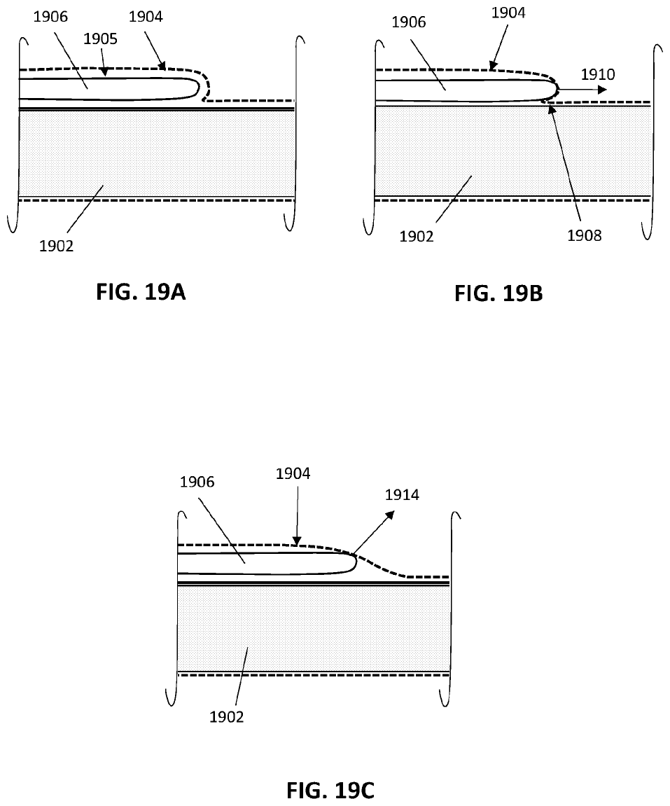

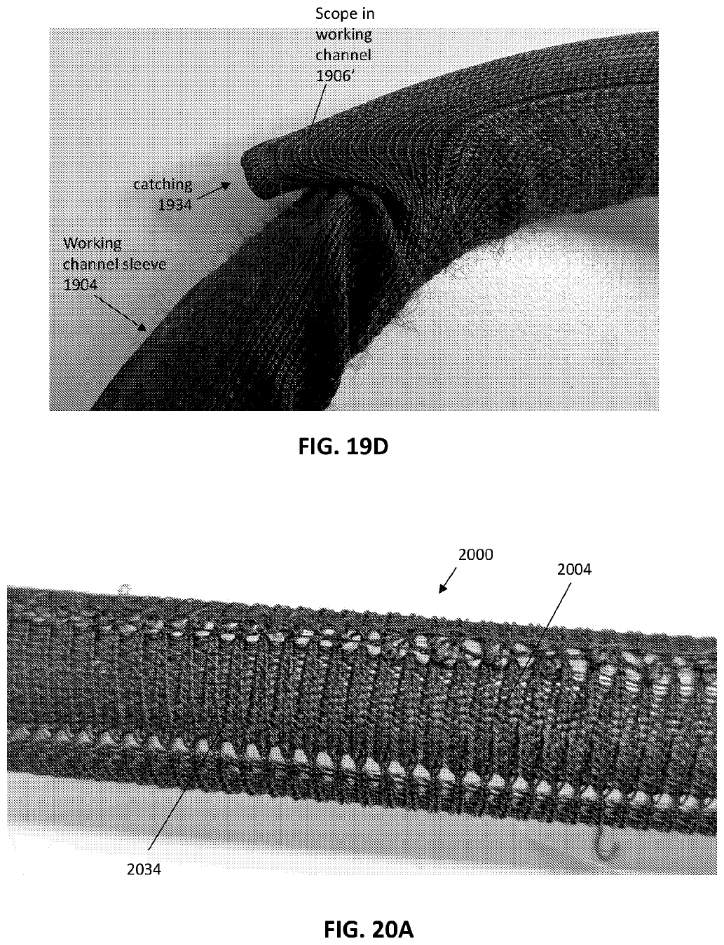

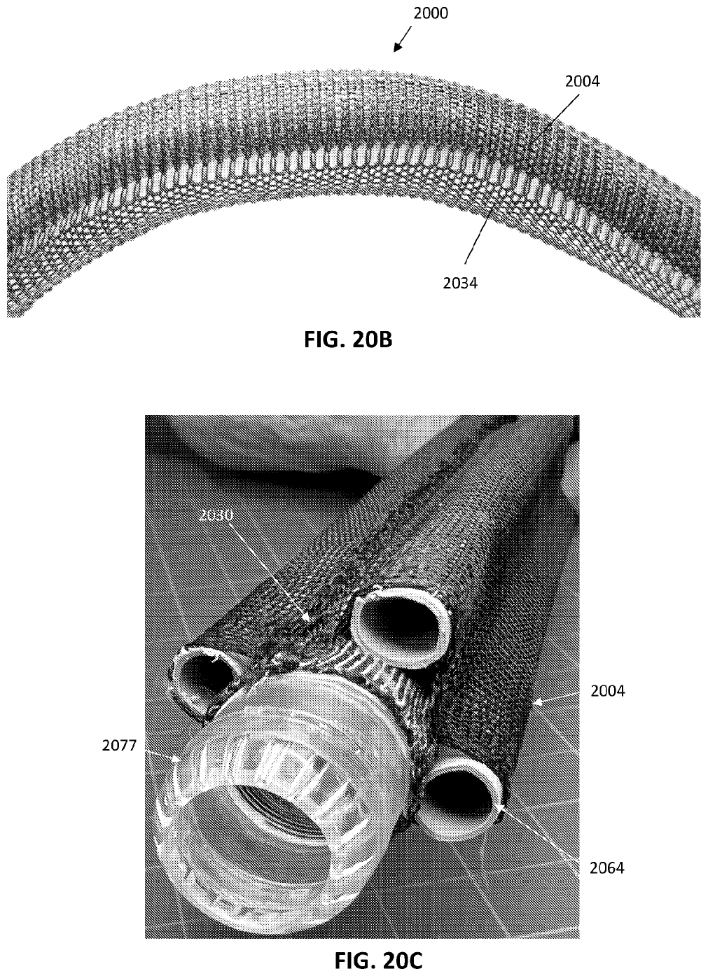

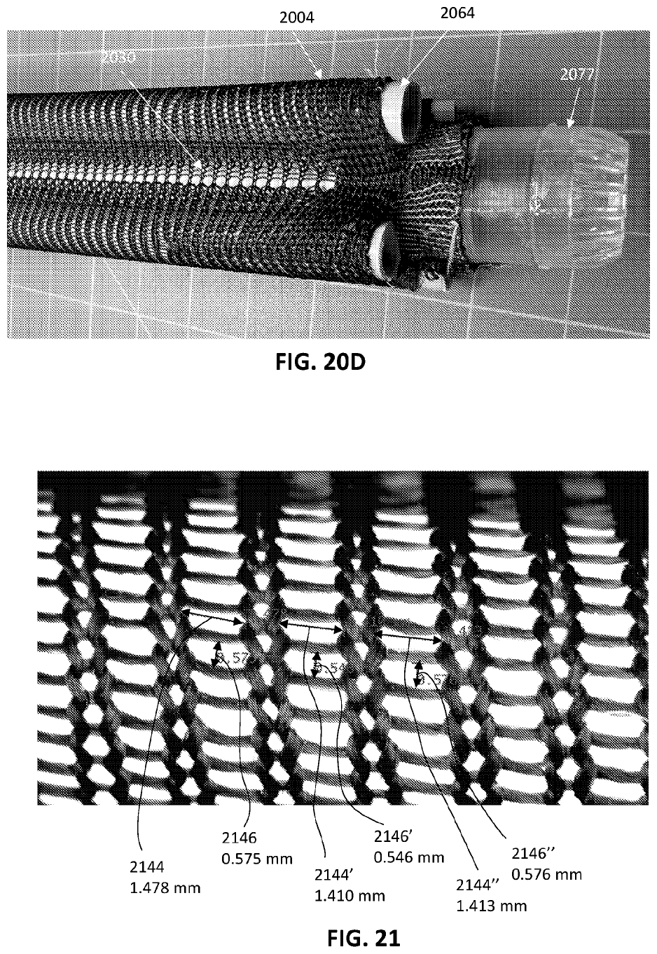

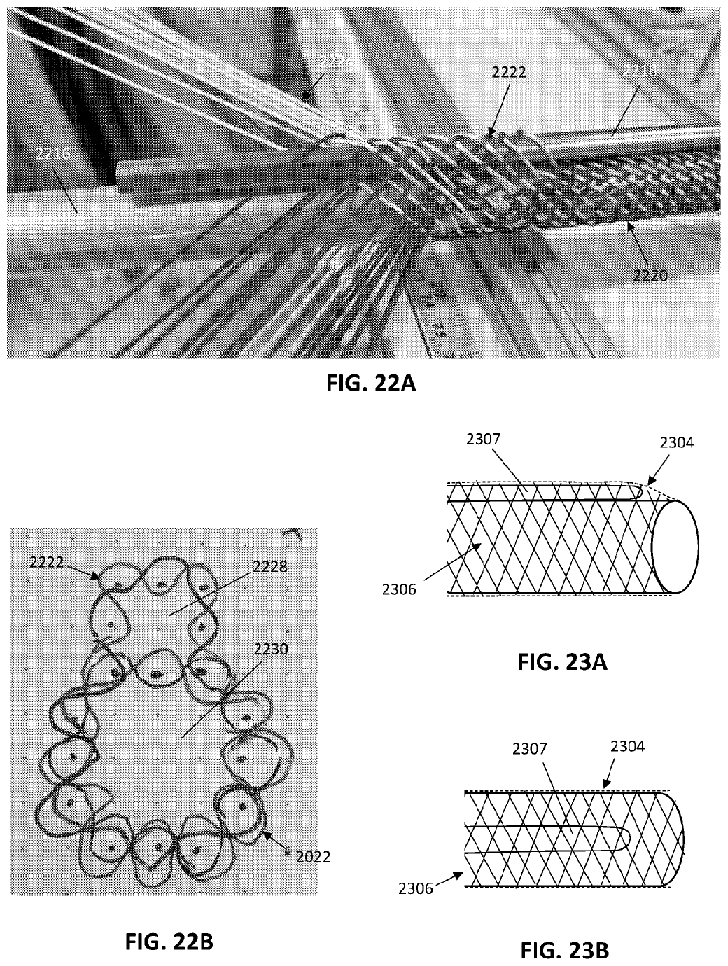

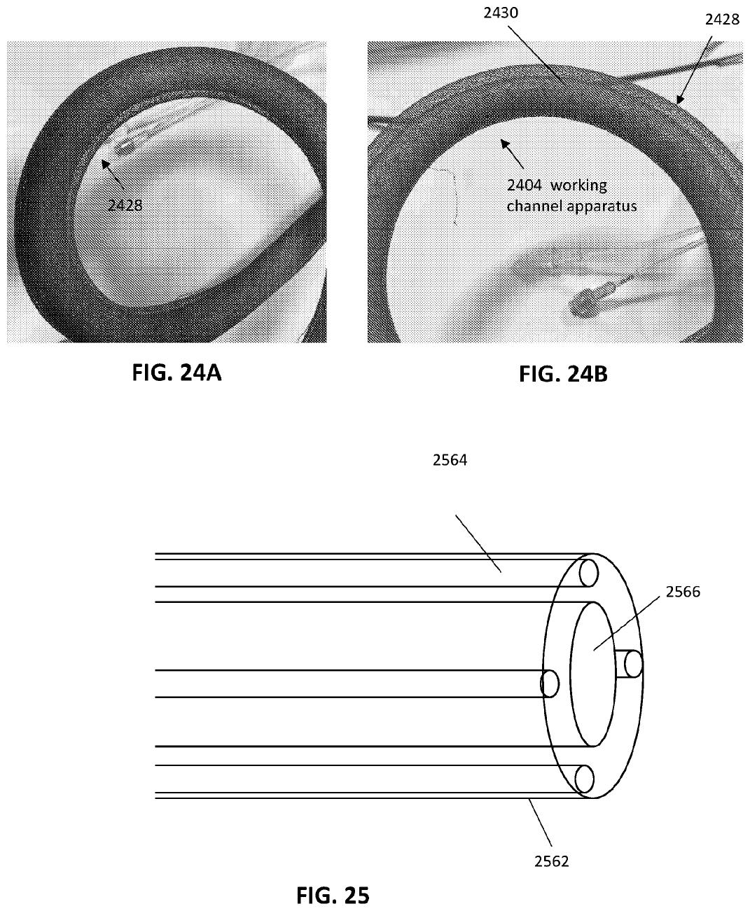

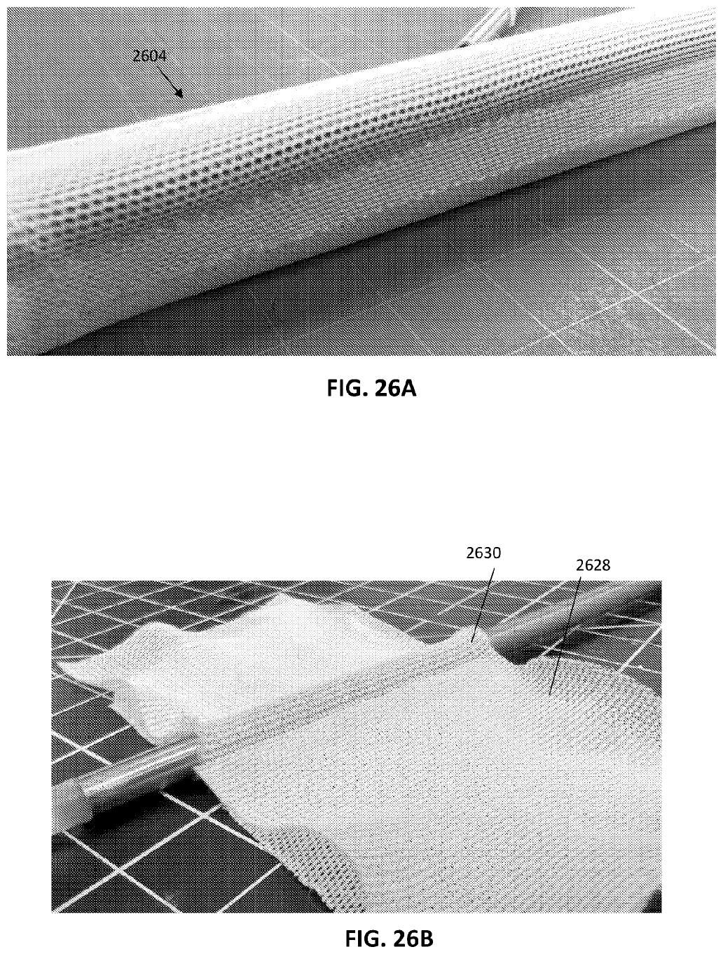

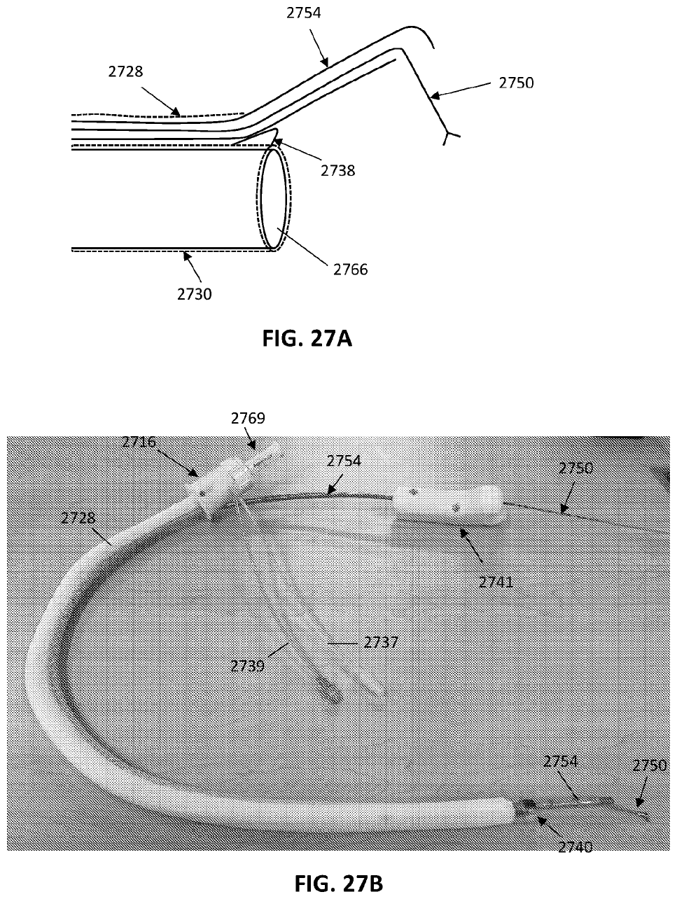

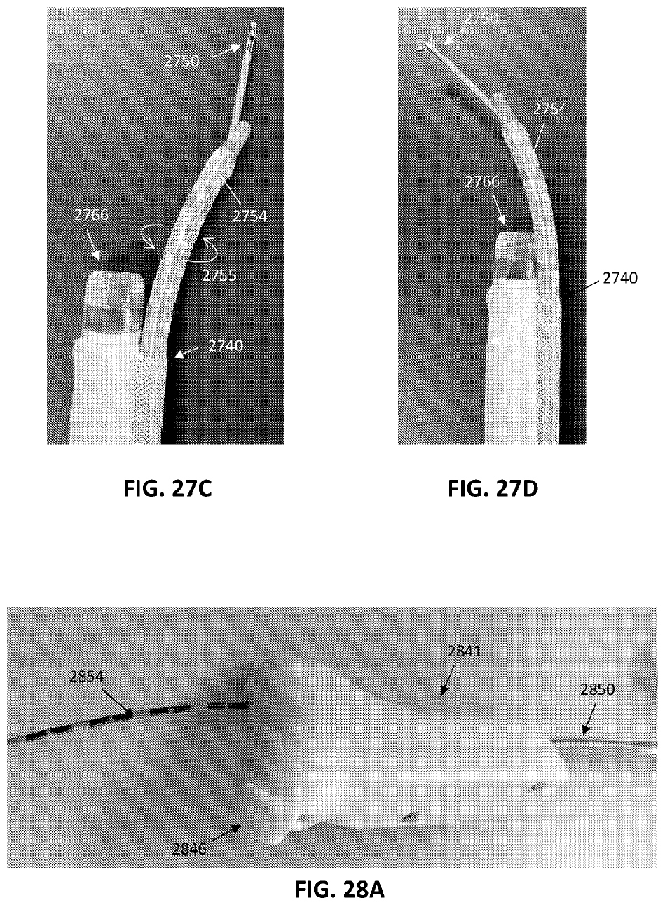

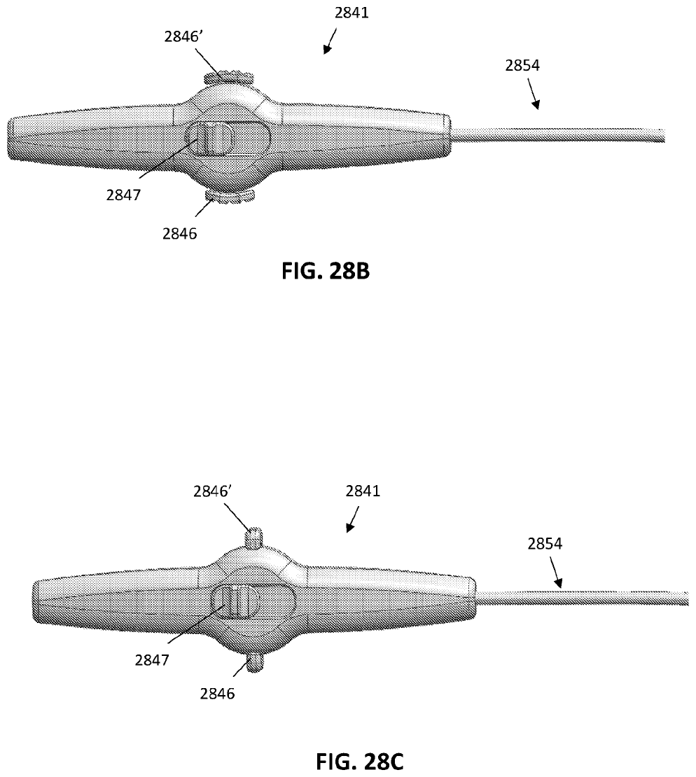

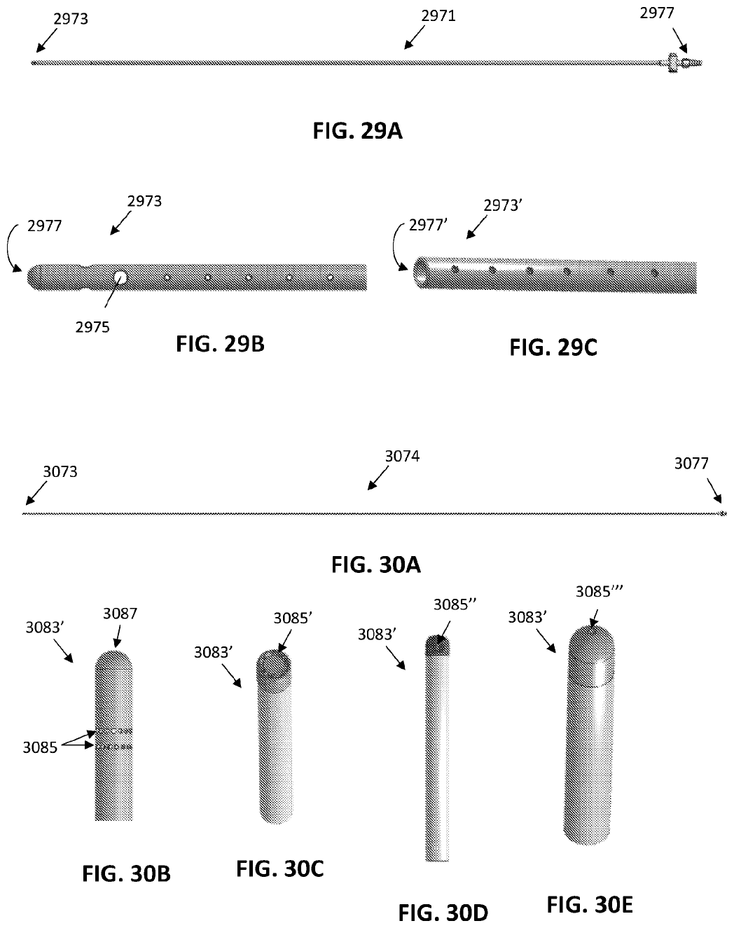

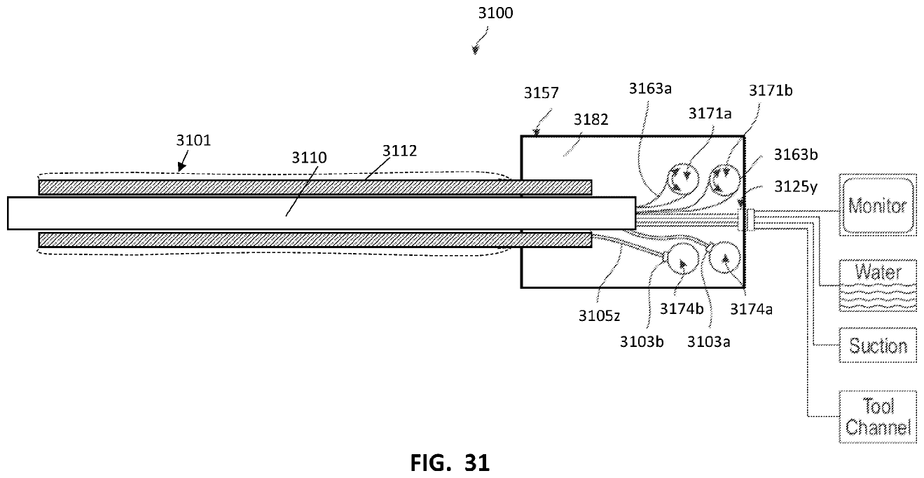

device to which a tube comprising a plurality of external, expandable working channels are coupled. FIG. 9A shows the channels in a collapsed configuration, while FIG. 9B shows the channels in an expanded configuration, driven open by a liner insert tube. FIG. 9C shows an enlarged view of the introducer region or guides for insertion of tubes into the external working channels of an apparatus as described herein. FIG. 9D shows an example of a liner insert tube that is of non-circular outer geometry. FIG. 9E shows a section through a portion of an apparatus including external and expandable working channels.

FIG. 10A shows an example of a liner insert tube.

FIG. 10B shows the distal and proximal ends of the liner insert tube of FIG. 10A.

FIG. 10C shows the distal end of the liner insert tube of FIG. 10A illustrating a deflectable instrument emerging from the distal end of the liner insert tube.

FIGS. 11A-11C illustrate one example of a system including a tube having an expandable external channel coupled to an elongate medical device (in this example, a rigidizing overtube). FIG. 11A shows the proximal end of the elongate medical device and an introducer for introducing a tool and/or a tool liner tube into the expandable external channel, with an instrument entering the liner. FIG. 11B shows a distal end of the tube and expandable external channel at a distal end of the elongate medical device, with the tool liner tube extending distally out of the working channel. FIG. 11C shows a tool extending from the tool liner tube at a distal end of the elongate medical device.

FIG. 11D is a schematic cross-section of a tool liner tube including an engaged deflector extending distally out of the elongate medical device.

FIGS. 11E-11G show one example of a tool liner tube.

FIGS. 11H-11L illustrate a tool inserted through the tool liner tube shown in FIGS. 11D-11G.

FIG. 12A is a scanning electron micrograph showing knit fibers of one example of a tube having expandable external channels.

FIG. 12B is example of a knit pattern of a section of an apparatus as described herein (shown with the tubular structure flattened).

FIG. 13 schematically illustrates one example of a method of operating an apparatus as described herein.

FIGS. 14A-14C illustrate one example of an elongate medical device onto which a tube having a plurality of external and expandible channels is coupled. FIG. 14A shows the overall system including the elongate medical device and tube with external working channels that are coded by a colored indicator (e.g. shape, pattern, alphanumeric, etc.). FIG. 14C is a view of a distal end of the apparatus of FIGS. 14A-14B, with two different instruments emerging. In FIG. 14B the tool is an aspiration catheter, and in FIG. 14C a liner insert tube with a deflector and a grasping instrument are shown.

FIGS. 15A-15B illustrate an example of a system including a plurality of external channels.

FIG. 15C shows the system of FIG. 15A with a variety of different tools extending from the external working channels.

FIGS. 16A-16E illustrate another example of a system as described herein, including a tool liner tube.

FIG. 17 shows an example of an elongate medical device (e.g., rigidizing device) having an outer tube with a plurality of expandable channels extending over an outer surface of the elongate medical device.

FIG. 18 is an example of the view seen from the endoscope, with the overtube distal end quadrant indicators (e.g., colors) corresponding to the different external channels shown.

FIGS. 19A-19D illustrate examples of challenges (e.g., catching, pocketing, pouching, drag, etc.) that may be addressed by the methods and apparatuses described herein.

FIGS. 20A-20B illustrate examples of knitted external working channel apparatuses, in this example, horizontal knit apparatuses, having four external working channels.

FIGS. 20C and 20D show examples of knitted external working channel apparatuses similar to those shown in FIGS. 20A-20B with the external working channels shown expanded by a tube inserted therein.

FIG. 21 illustrates an example of the pore (gap or opening) sizes of one example of a horizontal knitted external working channel assembly/apparatus.

FIG. 22A illustrates one example of a method of forming an external working channel assembly as a multi-lumen braid.

FIG. 22B schematically illustrates an example of an external working channel apparatus/assembly configured as a multi-lumen braid.

FIGS. 23A-23B schematically illustrate an example of a braided external working channel assembly having non-elastic filaments that are oriented to provide axial tension to prevent catching of a tool inserted into the working channel.

FIGS. 24A-24B show an example of an external working channel apparatus formed as a double layered braid.

FIG. 25 illustrates another example of a working channel assembly.

FIG. 26A shows an example of a sewn working channel assembly.

FIG. 26B shows an example of a partially constructed assembly formed from a thermoplastic polyurethane material that may be thermally processed.

FIG. 27A schematically illustrates a working channel assembly/apparatus including a deflector at a distal end as well as a matched pair of tools inserted through a working channel as described herein.

FIG. 27B shows an example of a working channel assembly including a rigidizing and flexible elongate member as well as a matched pair of tools.

FIGS. 27C and 27D show examples of the distal end of an apparatus including steering of an inner tool (e.g., a grasper tool) by a matched steerable tool.

FIG. 28A shows one example of a handle of a steerable tool of a matched set of tools for use with an external channel as described herein.

FIGS. 28B and 28C illustrate operation of a handle portion of a steerable tool (e.g. a steerable liner) as described herein.

FIGS. 29A-29C illustrate one example of a suction catheter tool that may be used with any of these apparatuses and methods described herein.

FIGS. 30A-30E illustrate an example of an irrigation catheter tool that may be used with any of these apparatuses and methods described herein.

FIG. 31 illustrates an example of a robotic system including an external working channel sleeve apparatus as described herein.

### DETAILED DESCRIPTION

In general, described herein are apparatuses (e.g., systems, devices, etc.) including one or more external working channels. These external working channels may be part of the elongate medical device, or they may be part of a working channel sleeve apparatuses that may be worn, applied or attached to the elongate medical device. Any of the external working channels may be expandable working channels. The elongate medical device may be part of the apparatus (e.g., part of the working channel sleeve apparatus) or may be separate from it.

Described herein are external working channel apparatuses that may extend over longer lengths (e.g., longer than 12 inches, 14 inches, 16 inches, 18 inches, 20 inches, 22 inches, 24 inches, 30 inches, 48 inches, 60 inches, 72 inches, 84 inches, etc.) without binding or catching. These external working channel apparatuses may not significantly reduce the overall flexibility of the tubular member (e.g., catheter, endoscope, etc.) onto which they are attached. These external working channel apparatuses may also provide multiple working channels, and may be effectively used on devices adopting a large amount of curvature. In general, the external working channel apparatuses may achieve advantages not possible with more traditional external (or internal) working channel systems.

Surprisingly, these apparatuses may be formed of either fully non-elastic materials or partially of non-elastic materials. In some examples the regions of the external working channel apparatuses forming the “expandable” working channel(s) may be formed primarily of non-elastic filaments or fabric that may expand and contract based on mechanical movement, e.g., sliding, rather than elastomeric properties of the filaments. This is in sharp contrast with most, if not all, previously described “expandable” external working channel apparatuses, which instead on elasticized systems to provide expansion and collapse of the working channels, which may help to reduce the passive size of the channels. However, as described herein, the use of elastic materials in this manner may result in a radially downward force vector (e.g., a normal force) only on the channel, but on the tools within. The resulting, relatively high, normal force results in a significant drag, which becomes particularly problematic at longer lengths and curvatures, for example when inserting a tool having a relatively long length over a tortuous path, as the resulting capstan drag rises exponentially with curvature. In contrast, the methods and apparatuses described herein may instead rely on the use relatively non-elastic materials that are configured to instead slide over each other to mechanically expand and contract.

External working channels have also proven particularly difficult to implement on very flexible or dynamic apparatuses, such as flexible and/or rigidizing elongate members. In addition to potentially limiting the flexibility. One potential solution is to permit movement between the external working channel apparatus (e.g. the “core” portion of an external working channel assembly) and the elongate tubular member (e.g., catheter, endoscope, etc.) over which the external working channel assembly is arranged. For example, the external working channel assembly may be attached to the elongate tubular member at the proximal and distal ends, but may be unattached, or in some examples, partially attached, or loosely attached, or only intermittently attached along all or regions of the length of the elongate tubular member. However, as described herein, it may be particularly important to control the friction between the external working channel apparatus (e.g., the core region) and the elongate tubular member (e.g., the catheter, endoscope, etc.) in order to avoid gathering and/or binding up of the external working channel apparatus on the elongate tubular member, particularly when inserting though a body lumen that may apply a drag force against the external working channel apparatus.

In some examples the engagement between the outer surface of the elongate tubular member and the inner surface of the core region may be configured to provide a shear engagement that effectively increases the frictional force between the two, e.g., allowing but limiting sliding. For example, the core region may sit in or on an indented or textured outer surface of the outer tube. An indented outer surface may be formed into the outer surface of the elongate tubular member that matches the inner surface (e.g., pores) of the core region.

When inserting one or more tools through the external working channels it may also be important to prevent snagging, catching, pouching, stalling or puncture of the external working channel, in addition to generally reducing the internal drag within the channel. For example, as a tool is passed within an external working channels, the tip of the tool (which may have a variety of shapes) may stall, snag, or even puncture the working channel. Stalling (stall out) may occur when the tool cannot be further inserted, e.g., when drag forces on the tool, e.g., from the walls of the working channel, become sufficiently large so that further insertion is not possible or is not easily performed. The external working channel apparatuses described herein may prevent or reduce these problems using one or more features that enhance the overall performance of the external working channel apparatus, including the use of non-elastic filaments to form the external working channels, controlling the orientation of the filaments, controlling the dry/wet properties of the filaments, controlling the size ranges of the filaments, using coatings and/or liners within the working channels, and forming the working channels from appropriately oriented and structured knitted, braided and/or sewn materials, e.g., in some cases to permit local siding of the filaments forming the channels, and/or to permit anisotropic stretching. The external working channel apparatuses described herein may also be configured so that the overall elasticity of the apparatus may vary along the length of the apparatus in a controlled manner. Each of these factors are improvements that may be included, and any of these features may be combined with one or more (or all) of the other features. However, it should be understood that it is not necessary that an external working channel apparatus include all of these features.

As described, the external working channel apparatuses described herein may be used with elongate members, and particularly (but not limited to) longer, and/or flexible elongate members. These external working channel apparatuses (e.g., devices, systems, etc.) may be used with a catheter, an endoscope (including, but not limited to colonoscopes, bronchoscope, colposcope, cystoscope, esophagoscope, gastroscope, laparoscope, thoracoscope, enteroscope, etc.), overtube, etc. These apparatuses and methods may be used with a robotic system, including a robotically controlled endoscope. Robotic systems may be steered and/or advanced robotically. In some examples, the robotic system may control the operation (e.g., advancing, retracting, and/or actuating) of one or more tools to be used within an external working channel, including any of the tools or tool pairs described herein.

The external working channels described herein may be particularly beneficial when used with elongate members (e.g., catheters, endoscopes, overtubes, etc.) that are rigidizing, including dynamically rigidizing, and may convert from a highly flexible member to a relatively rigid member, e.g., by the application of external pressure. A rigidizing elongate member may provide support and stability, which may prevent unintended or undesirable movement of the rigidizing member when inserting or operating a tool through the working channel.

In particular the working channel sleeve apparatuses described herein may include a tube having one or more external working channels that are configured to aid in transporting a medical instrument (e.g., a tool or tool liner) through a body, including along a curved or looped pathway. Any appropriate elongate medical device may be used and/or may form part of the apparatus, including (but not limited to) rigidizing elongate medical devices, such as those described in PCT/US2021/034292, filed on May 26, 2021, entitled “RIGIDIZING DEVICES”, the entirety of which is incorporated by reference herein. The tubes including the expandable external working channels described herein may include fabric tubes, such as a knitted, woven, braided, etc. In some examples the tube are non-woven tubes, such as laminate tubes or tubes formed of an elastic sheet, etc., such as a thin elastomer sheet that has numerous cut-outs such forming pores or openings therethrough.

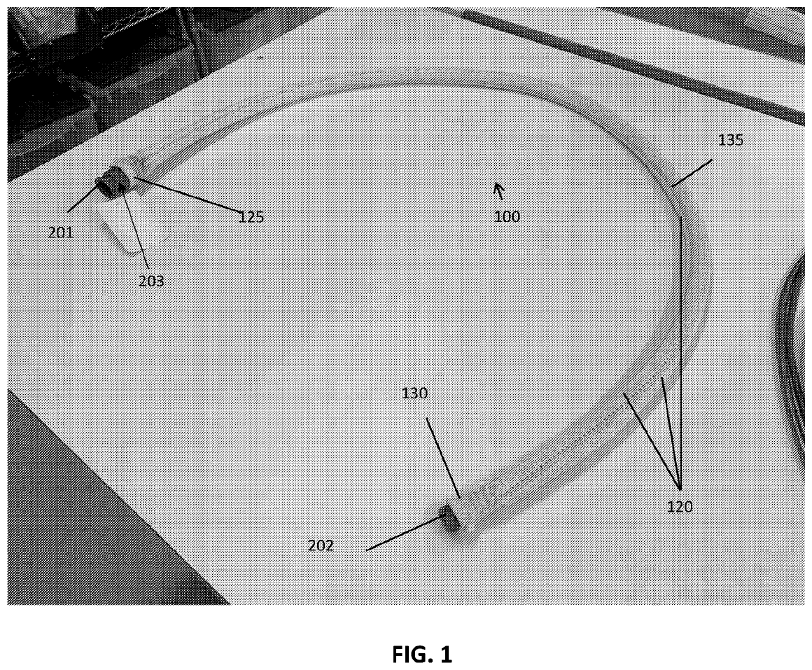

FIG. 1 shows an example of a working channel sleeve apparatus 100 including a plurality of external working channels and an inner tube. The external working channels for use with the elongate medical device can be formed as part of (e.g., integrated into) an inner tube 135 (e.g., fabric tube) extending between a proximal end region 125 and a distal end region 130 of the elongate medical device. The proximal end region 125 may be exteriorly accessible when the external expandable working channels 120 are in use and the distal end region 202 is partially or substantially disposed within a vessel. The tubular material forming the inner tube may be expandable and/or flexible to selectively accommodate travel through angled or curved passages. A plurality of working channels 120 may be formed on the tube, and in particular, may be formed of one or more filaments that are knitted, woven and/or braided integrally with the tube. The inner tube may comprise a mesh material. In some examples the working channel sleeve apparatus includes both the inner tube and a plurality of external working channels, as shown in FIG. 1.

The tube 135 of the working channel sleeve apparatus shown in FIG. 1 may include a lumen that may extend from the proximal end region to the distal end region. The tube 135 may be generally flexible in a relaxed configuration and may be collapsible when nothing is positioned therein, e.g., when not applied or attached over an elongate medical device 200. As mentioned, one or more channels (e.g., expandable working channels 120) may be formed and/or positioned around the exterior surface of the main tube 135.

The expandable working channels 120 may extend longitudinally from the proximal end of the tube 125 to the distal end of the tube 130 (or from the proximal end region to the distal end region). A working channel 120 may have a lumen extending the entire length of the working channel 120 allowing for passage of objects that may be slide through the working channel 120 into the proximal end 125 of the working channels 120 and out through the distal end 130 of the working channel 120.

Thus, in any of the working channel sleeve apparatuses described herein, the working channels may be advantageously formed of one or more fibers (e.g., knitted, woven, braided, etc. fibers) and may be expandable (e.g., stretchable). These external working channels may be configured to lay flat (e.g., as “layflat”) against the outer surface of the elongate medical device until expanded by inserting a tool or tool inserted. In particular, these apparatuses may include working channels that may be co-formed as a knit, weave and/or braid, with the tubular body 135. The tubular body may be a fabric tube, which includes knit, woven, or braided tubular bodies. In some examples the working channel sleeve apparatus is formed of a mesh material (e.g., knitted or woven) so that both the inner tube member and the external working channels are stretchable and flexible. The working channel sleeve apparatus may be arranged relative to the elongate medical device so the entire working channel sleeve apparatus/assembly (including the tube 135 and the one or more working channels 120) may be formed as a knitted tube or sleeve that is sized to fit over the elongate medical device so that the working channels may be expanded and used to pass one or more medical tools and/or a liner insert tube that may be inserted to form a more open and lubricious channel for a tool.

The length of a working channel 120 of a working channel sleeve apparatus may be approximately the same length of the inner tube (e.g., tube 135) from the proximal end or tip to the distal end or tip of the working channel sleeve apparatus. Alternatively, each of the expandable working channels 120 may have a length less than that of the tube 135. For example, a working channel 120 may extend longitudinally along the outer surface of the tube 135 whereby a proximal end of the working channel 120 is positioned on the exterior surface of the tube 135 between the proximal end and the distal end of the tube 135. Likewise, the distal end of the working channel may be positioned on the exterior surface of the tube 135 between the distal end of the tube 135 and the proximal end of that working channel.

The working channel(s) 120 on the exterior of the tube 135 may begin at or substantially near one another and may being at or near the proximal end of the tube 125. The distal end 130 of the working channel(s) 120 may be positioned at any point along the tube 135 between the proximal end 125 and the distal end of the tube 135, including where the distal end of any other working channel(s) and the distal end of the tube 135 terminate, or substantially nearby.

As mentioned, the external and expandable working channel(s) 120 may be formed as part of the tube 135 or in some examples may be affixed to the tube 135 of the working channel sleeve apparatus. A portion of the exterior surface of the working channel(s) 120 may contact the exterior surface of the tube 135 or may be integrally formed as part of the exterior surface of the tube 135. Expandable working channels 120 may have a collapsible cross-sectional geometry. The working channel(s) may have an interior surface defining the lumen of the working channel, and an exterior surface of the working channel material may be exposed and visible as the outer most layer of the system 100. The interior of the working channels may be lubricious (e.g., low friction).

In some of the examples described herein, the system 100 including the elongate medical device and working channel sleeve apparatus (e.g., inner tube and external working channels) may be equivalently referred to herein as an external working channel system 100 or external working channel device. In some examples, for convenience these apparatuses may be referred to herein as working channel sleeve apparatuses and may include the elongate medical device.

As mentioned, the expandable working channels 120 and tube 135 may be formed of non-elastic material, or in some examples, of a hybrid elastic and lubricious material 210 (described in greater details in FIG. 7, below) and may be formed by one or more filaments having desired properties. In particular, the one or more filament(s) may be fibers, a network of woven fibers, a yard, a thread, or a combination thereof. In some examples the filaments may have an elastic inner region (e.g., core) with a lubricious outer region (e.g., coating, wrapping, etc.). The elastic core may be sufficiently elastic to allow stretching of the knitted, woven or braided material forming the working channels. For example, the elasticity of the fibers may allow for stretching and dynamic changes in length of the fibers as the external working channel system 100 is manipulated. The elastic core may be any appropriate biocompatible elastic material, such as a silicone, spandex, biocompatible polyurethane elastomers and/or biocompatible copolymer elastomers. The lubricous outer region may be formed of any appropriate biocompatible lubricous material, such as polypropylene, a polyethylene, or polyester, or a polytetrafluoroethylene (e.g., TEFLON). The materials may be used in totality or partially, and/or combined with other materials.

FIG. 7 schematically illustrates an example of a hybrid material that is both elastic and lubricious and may be used to form the tube and/or expandable external working channels. In this example, a double covered filament (e.g., “yarn”) consisting of an elastic core 736 wrapped with two counter-wound layers 738, 740 of more lubricious fibers 734 is shown. In FIG. 7 the hybrid material includes an elastic (e.g., elastomeric) core 736 that is wrapped by a double wrapping of a less elastic, but more lubricous fiber 738. The elastic core may be a single fiber or multiple elastic fibers, which may be arranged in parallel or may be twisted, braided, etc. In some examples a single covering 738 of the lubricous fiber may be wrapped around the elastic core; in some examples a double 738, 740 or more (e.g., triple, etc.) overlapping wrapping of the less elastic but more lubricous material may be used. Although a single covering is possible, a double covering may have more reliable coverage.

In another example, the elastic element and the lubricious element are knitted, woven, or braided together in such a manner that the elastomeric material (e.g., “core”) may not be wrapped by a lubricious material completely, but nevertheless does not add to the drag of a sliding member because the elastomeric member is positioned relative to the working channel away from the sliding surface. As such, the elastomeric material (e.g., “core”) may provide the requisite elasticity, but does not significantly increase drag.

The tube 135 may be formed of the same material as the expandable working channels or may be formed of a separate material that may be more or less elastic and/or more or less lubricious. For example, the lumen 140 of the tube 135 that is configured to fit over the elongate medical device may have an adjustable circumference, radius or diameter to accommodate the elongate medical device (e.g., a rigidizing tube) to which it is applied over by being sufficiently elastic. Similarly, each of the expandable working channels may have an expandable lumen 126 with an adjustable circumference, diameter or radius to accept a liner insert tool and/or to directly accommodate tools of different dimensions. In some example, the filaments can have a low-friction coating and/or a low-friction wrapping around the elastic core. The coating material may increase the lubricity of the fiber and may reduce friction when a fiber contacts another surface (e.g., a sliding device or the interior of a body vessel). The use of a low friction filament material similarly ensures reduced friction relative to adjacent sliding materials or surfaces. The lubricious wrapping and/or coating can be a material such as polypropylene or Teflon or a combination thereof. In some examples, the one or more filaments forming the expandable working channels may be wrapped with multiple layers of the lubricious material. In some examples the filament(s) may include an elastic core that is wrapped (e.g., coil wrapped) by a radially attached wrap material (e.g., a polytetrafluoroethylene material) to the elastic core (e.g., a silicone core, a spandex core, a urethane core, etc.). The wrap can include the lubricious material and each filament may have more than one layer of wrap.

In some examples, the forming the working channel(s) 210 and/or the tube can compress against an elongate medical device as the filaments are stretched to accommodate the changes in shape (bending, expand/contract, etc.) of the elongate medical device, allowing the tube and/or working channels to compress or retract. The compression/expansion of the flexible tube and working channels on the elongate medical device may be sufficient to prevent warping, rippling, kinking, wrinkling, buckling, or bunching of the tube and/or working channels as the rigidizing device assembly is advanced through a vessel. The weave and network of filaments can be one example of a knit or woven material forming an expandable working channel 120 and the tube 135. The tube 135 can form a continuous lumen 140 into which the elongate medical device fits. Additionally, the lumen 126 of the working channels may also be formed of the same (or a different) filament or type of filaments.

In any of the tubes having working channels described herein may be knitted, braided and/or woven as described above. The resulting may form pores between the filament(s) forming the fabric. As used in this example “pores” includes windows, openings, gaps, spaces, etc. between filament(s) forming the (e.g., weave, knit, etc.). In general, the tubes described herein may have a pore size that may vary as the working channel is expanded or collapsed. The pores may change shape (i.e., compress, be reduced, expand, or extend) as the working channel shape changes. Smaller pore sizes may prevent snagging or catching of a tool within the expandable working channel(s). The optimal sizing of the pores may depend on the material, including filament size, pore percentage, size of the spacing of pores, pore diameters, etc. For example, in some examples it is beneficial to have a porosity of greater than <80% (less than 75%, less than 70%, less than 65%, less than 60%, less than 55%, less than 50%, etc.,) when the expandable working channel not expanded (“opened”). The pore size of the flexible tubular member may range from 0.05 mm to 4 mm. In general, the flexible tubular member may have a variety of pore sizes and shapes along its length.

The knit may be formed using between 1-4 filament ends, having fibers of Denier (thickness) of between about 10-200. The knitting machine gauge may be between about 10/14 and 16/18.

The lumen of the external working channels may be defined by an exterior surface of the tube 135 and an interior surface of channel material such that the channel material is connected to the exterior surface of the tube 135. When a liner insert tube and/or a tool is passed through an external working channel (which may be an expandable working channel), the material forming the external working channel may expand even while the exterior surface of the tube 135 within the channel lumen does not. Alternatively, in some examples an expandable working channel may have an interior surface of a knitted, woven or braided filament(s) that are separate from the filaments forming the tube and may be affixed to the exterior surface of the tube 135.

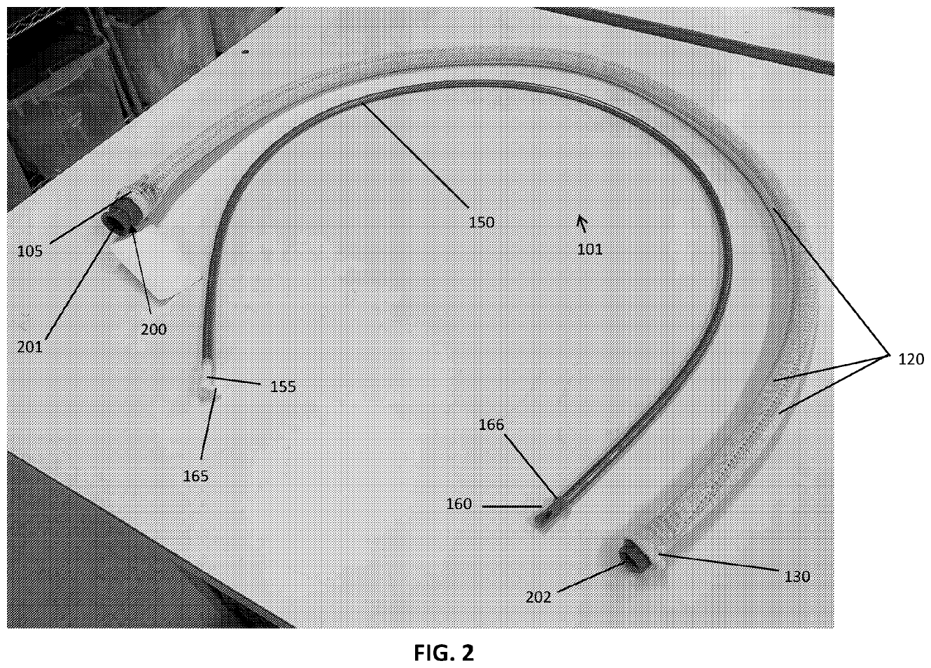

FIG. 2 shows an example of a working channel sleeve apparatus 101 similar to that shown in FIG. 1 including an elongate medical device 200 (in this example a rigidizing tube) installed through the tube 135 having a plurality of expandable working channels 120. The expandable working channels 120 are visible around the exterior surface of the tube 135. This system also includes a liner insert tube 150 that has a length that is approximately the same as the distance between the proximal end region and the distal end region of the tube and/or the elongate medical device. In some examples, because the distance along the outside of the tube when it is curved may be different than the distance along the inside of the tube when it is curved, the liner (e.g., the liner insert tube 150) may be longer than the midline path length of the apparatus, such that the liner insert tube can buckle to accommodate when on the inside curve yet may still be long enough when extending along the outside curve of the elongate medical tool. The liner insert tube 150 may be configured to fit into the lumen of an expandable working channel and may extend from the proximal end to the distal end. In some examples the distal end of the liner insert tube 150 may engage with the distal end of the tube and/or the distal end of the elongate medical device. The liner insert tube 150 may be configured to receive one or more tools or instruments passed therethrough, while the liner insert tube 150 holds the working channel open.

In some examples the liner insert tube 150 can have a substantially thin wall around a lumen. The liner insert tube 150 may have an interior surface surrounding the lumen and an exterior surface that may be in contact with an interior surface of a working channel 120. The liner insert tube 150 may resist compression by the expandable working channel to facilitate advancement through the external working channel device 100. Additionally, the liner insert tube 150 may be bendable and can proceed along a path defined by an interior of a channel 120 that is curved or bent based on the path of the elongate medical device within the body. The external working channel is disposed on the same path as the elongate medical device 200, and the liner insert tube 150 may be inserted after the distal end of the elongate medical device is positioned near the target tissue region to be treated or examined. Advantageously, the elongate medical device may be positioned within the body with a small profile that is later expanded to a larger profile to fit one or more tools by inserting and engaging the liner insert tube 150 in the expandable working channel. Each liner insert tube 150 may have a tapered distal end with one or more engagers (e.g., engagement elements) that may engage with a distal end region of the external working channel, the tube, and/or the elongate medical device. The engagement element 165 of the tool liner can be configured to communication with a complimentary engagement region or element on either the elongate medical device or the tube, or both. In some examples, a proximal engager (engagement element 165) at the proximal end of the liner insert tube 150 is configured to engage the extremal geometry of the tube, and/or external working channel and/or elongate medical device. As mentioned, the distal end of the liner insert tube 160 may also or alternatively have a distal engager (e.g., distal engagement element 166) configured to engage or otherwise communicate with a distal end region of the tube and/or elongate medical device. These engagers may be of different shapes, including wing-like.

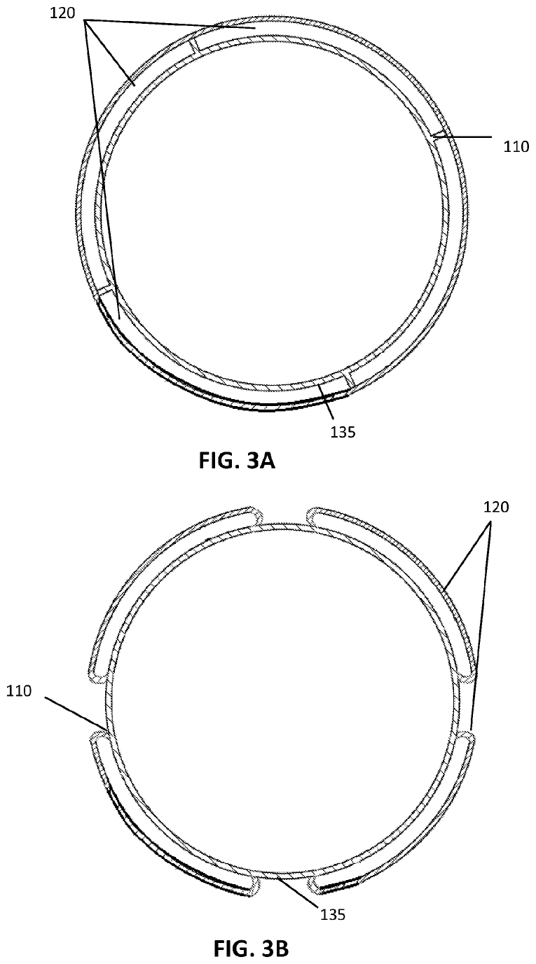

According to some examples, the material forming the external working channels 120 may form a continuous circumference whereby a region of the outer circumference is affixed to the exterior surface of the tube 135. Accordingly, the portion of the tube 135 in forming or in communication with the outside of a working channel may be at a single point up to half of the circumference of the working channel. The amount of the working channel continuous with the tube 135 may be described in a percentage of the circumference of the working channel. For example, 1%, 5%, 10%, 15%, 20%, 25%, 30%, 35%, 40%, 45%, 50%, or more of the working channel circumference may be formed by the tube 135. In a relaxed configuration, the cross-sectional area of a working channel may be collapsed relative to the working channel when a liner insert tube or medical tool is inserted therein. The working channels may be formed of discrete region along an outer surface of the tube (as shown in FIG. 3B) or they may extend over the entire outer surface of the tube (as shown in FIG. 3A). In some examples the working channels may be integral with the tube. In FIG. 3A the external working channels 120 are formed of the same material (e.g., from the same filaments or sharing filaments) as the tube 135. In some examples (not shown) the working channels may be formed of separate filaments and may be attached to the tube 135 (e.g., by co-weaving, braiding, local stitches or bonds, or the like). In some examples, the working channels may be affixed to adjacent working channels as they share a common attachment points 110. Alternatively, the external working channels 120 may be formed integrally with (e.g., woven, braided or knitted into) the tube 135. In some examples, the external working channels may be radially spaced from each other and do not contact one another (see, e.g., FIG. 3B) and a portion of the exterior surface of the tube 135 is visible.

In general, as shown in FIGS. 3A and 3B, the external working channels may be in a collapsed configuration (e.g., lying flat on the outer surface of the elongate medical device) until something is inserted into the lumen or channel formed by the external working channel.

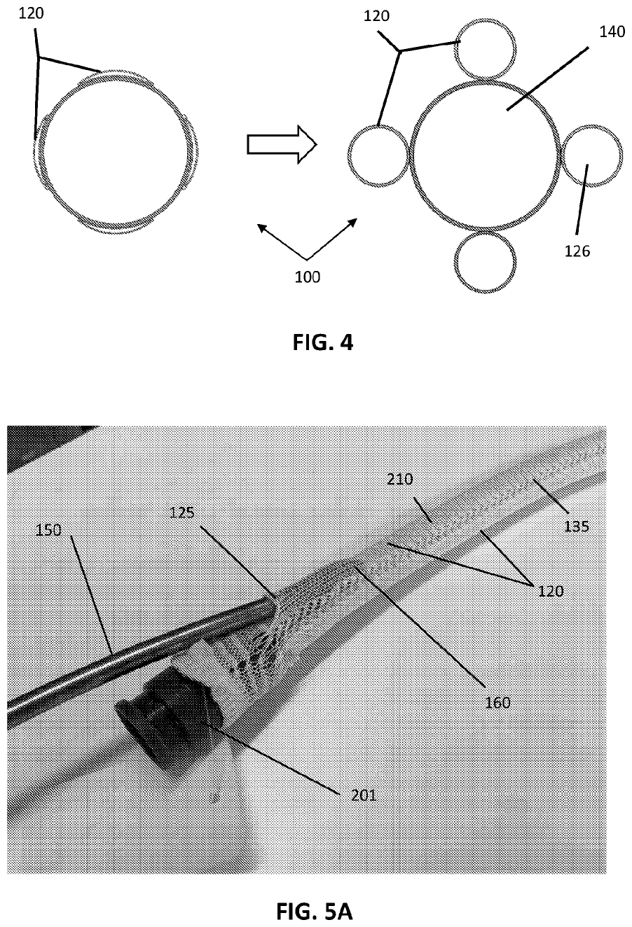

In general, the external working channels may increase their cross-sectional area to accept a tool and/or liner insert tube that may be slid or advanced into the working channel as illustrated by FIG. 4. In FIG. 4, the working channels 120 are shown in a collapsed configuration on the left, having a clear and empty lumen therethrough. The right side illustrates the working channels expanded, e.g., when a tool or liner insert tube is inserted therein. In some examples, in contrast to FIGS. 3A and 3B, the channels could be attached only locally, for example, at points or lines of tangency. The channels can be attached at their full contact circumference, or partial contact locations, all the way down to a single point of tangency. The flexible nature of the working channel may conform to the dimensions and geometry of the liner insert tube. A liner insert tube 150 may be inserted into the proximal end of the working channel. For example, a distal end 160 of the liner insert tube may be inserted into the proximal opening of the working channel 120 at the proximal end 125. A length of the liner insert tube may be slid into and through the working channel 120 towards the working channel distal end 130. The liner insert tube 150 may be advanced to any position within the length of the working channel 120. In particular, the liner insert tube may be fully inserted into the external working channel and may be locked into position. For example, the liner insert tube 150 may be advanced through the working channel lumen 126 such that the distal end 160 of the liner insert tube 150 extends beyond the distal end 130 of the working channel 120. The liner insert tube 150 in some examples may be inserted through the working channel 120 until the distal end of the liner insert tube 150 is substantially near or beyond the distal end of the tube 135. The distal end and/or proximal end of the liner insert tube may engage with the elongate medical device and/or tube.

According to any example described herein, the liner insert tube 150 can have a geometry (e.g., can be size and spaced) that is configured to allow one or more tools to be passed through the liner insert tube. It may be circular. In some examples a cross-sectional geometry of the liner insert tube lumen may provide for specific orientation of the tool as it is inserted and advanced through the tool liner. For example, the liner insert tube lumen may have a cross-sectional geometry that is semicircular or some form of a polygon corresponding to cross-sectional geometry of a tool passed therethrough. The liner insert tube may remain open even as the force compressing it by the collapsible working channel increases (e.g., when bent or highly curved).

In any of these examples, the liner insert tube may be visualized by a camera (e.g., a camera associated with the elongate medical device. For example, an imaging element may image the liner insert tube during a procedure as it emerges from the distal end region of the working channel. The imaging element can provide spatial awareness of the orientation of the liner insert tube inside a vessel where it may be difficult to image. It could be advantageous, therefore, to have the main endoscope image, and then a secondary endoscopic image, as provided by the endoscope that goes through the external working channel or liner. The second endoscope could also be useful for additional manipulation and grasping, including with a tool through its internal working channel.

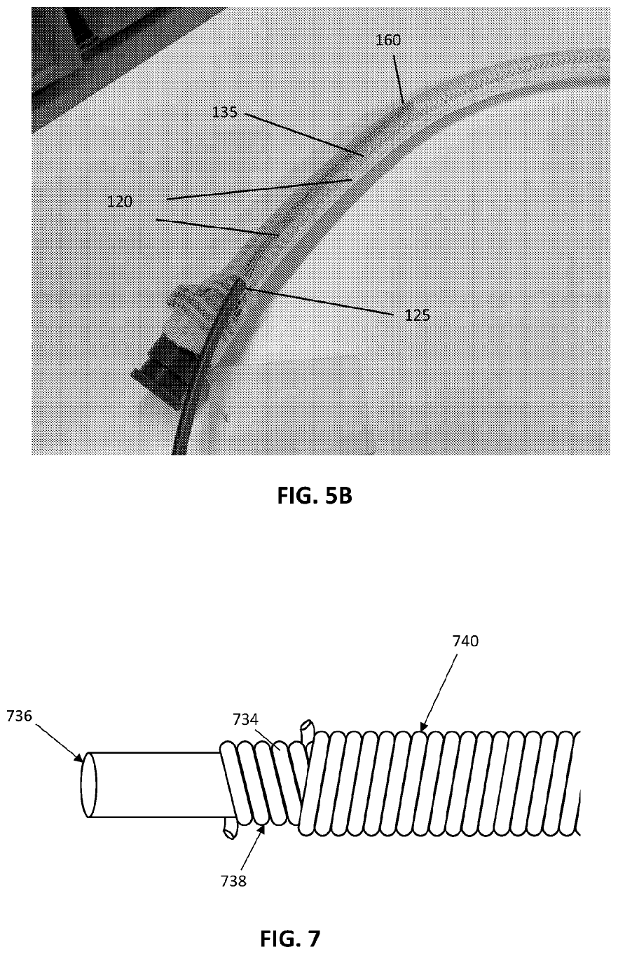

FIGS. 5A and 5B show views of the proximal end of the apparatus assembly (e.g., the elongate medical device and attached flexible tube including multiple external working channels). In FIG. 5A a liner insert tube 150 is shown inserted into a proximal end 125 of a working channel 120. The insertion of the liner insert tube 150 is near the proximal end 201 of the rigidizing device 200, as this may be the end of the system or assembly that would be most accessible during use. The distal end 160 of the liner insert tube 150 can be seen as within the working channel 120 as it is advanced through the apparatus through the knitted material forming the working channels in this example (as shown in FIGS. 5A and 5B). In this example the forming the working channel and tube is configured as a knitted filament that comprise the working channels and the tube 135. As shown, the working channel 120 expands as the liner insert tube 150 is first inserted into the proximal opening in FIG. 5A and continues to expand as the liner insert tube 150 is advanced from a proximal end 125 towards a distal end of the working channel 120.

The tubes including the working channels described herein may be configured to be worn over an elongate member device. For example the tube may receive an elongate medical device within the lumen 140 of the tube 135. The elongate medical devices can generally be long, thin, and hollow catheters, scopes, overtubes, or the like. In some examples the elongate medical device can transition from a flexible configuration (i.e., one that is relaxed, limp, or floppy) to a rigid configuration (i.e., one that is stiff and/or holds the shape it is in when it is rigidized). The apparatuses described herein may be particularly well suited for use with rigidizing devices but may be used with non-rigidizing devices. In some examples, a rigidizing device (also referred to as a selectively rigidizing device) may include a plurality of layers (e.g., coiled or reinforced layers, slip layers, braided layers, bladder layers and/or sealing sheaths) that can together form the wall of a rigidizing devices. The rigidizing devices can transition from the flexible configuration to the rigid configuration, for example, by applying a positive or negative pressure to the wall of the rigidizing device or within the wall of the rigidizing device. With the positive or negative pressure removed, the layers can easily shear or move relative to each other. With the positive or negative pressure applied, the layers can transition to a condition in which they exhibit substantially enhanced ability to resist shear, movement, bending, torque and buckling, thereby providing system rigidization. Any appropriate rigidizing member may be used, including rigidizing members that are not formed of layers and/or actuated by pressure (positive and/or negative pressure). For example, the rigidizable members described herein may refer to any appropriate rigidizing device, including members that may be rigidized by jamming particles, by phase change and/or shape memory alloys, by interlocking components (e.g., cables with discs or cones, etc.), EAP (electro-active polymers) or any other rigidizing mechanism.

The elongate medical devices that may be used as part of the apparatuses described herein (both rigidizing and non-rigidizing) may include catheters, sheaths, scopes (e.g., endoscopes), wires, overtubes (e.g., external working channels), trocars or laparoscopic instruments. Rigidizing devices can function as a separate add-on device or can be integrated into the body of catheters, sheaths, scopes, wires, or laparoscopic instruments.

According to some examples, the body with external working channels can be installed with or on a rigidizing device. The lumen 140 of the tube may receive a portion or segment of the rigidizing device 200. For example, a body of the rigidizing elongate device may be contained entirely within the tube 135 lumen. A distal end and proximal end of the rigidizing device 200 may be exposed as they extend beyond the length of the tube 135 of the external working channels. Alternatively, a length of the rigidizing device 200 from the distal tip to near the proximal end of the rigidizing device 200 may be within the external working channel.

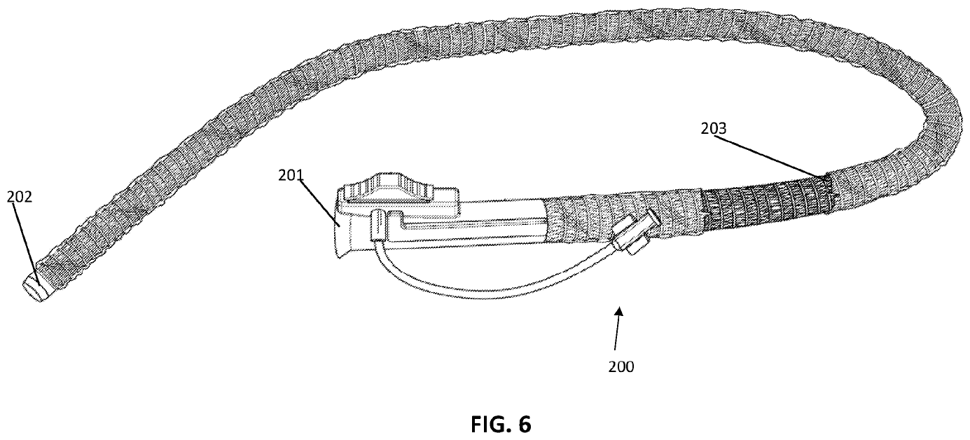

An elongate medical device configured as a rigidizing device 200 is shown in FIG. 6. The rigidizing device 200 in this example has a wall with a plurality of layers 203 including a braid layer, an outer layer (part of which is cut away to show the braid 203 thereunder), and an inner layer. The system further includes a handle 201 having a vacuum or pressure inlet to supply vacuum or pressure to the rigidizing device 200. An actuation element can be used to turn the vacuum or pressure on and off to thereby transition the rigidizing device 200 between flexible and rigid configurations. The distal tip 202 of the rigidizing device 200 can be smooth, flexible, and atraumatic to facilitate distal movement of the rigidizing device 200 through the body. Further, the tip can taper from the distal end to the proximal end to further facilitate distal movement of the rigidizing device 200 through the body.

The elongate medical device of FIG. 6 may be used as part of a system including one or more external working channels. For example, the external working channel tube 135 may cover an entire length of an outer surface of the rigidizing device 200. The tube and working channels may be applied over the rigidizing device, so that the tube may be fitted over the elongate body of the rigidizing device. In any of these examples, the tube and external working channels may be coupled to the elongate medical device either because of the elastic material forming the tube, which may contract down onto the outer surface, and/or because the tube may be attached at one or more points or regions (or along the entire length) of the outer surface of the elongate medical device. In some examples the tube forming the external working channels may be secured just at the distal end region or at both the distal end region and proximal end region. In some examples, the tube forming the external working channels may attach at various points or regions along the length of the elongate medical device (e.g., between about every cm and every 50 cm, between about every 15 cm and every 50 cm, between about every 20 cm and every 50 cm, between about every 25 cm and every 50 cm, between about every 30 cm and every 50 cm, etc.). In general, discrete attachments (and in some cases fewer attachments) may be preferred, to allow the tube and therefore the external working channels to adjust position slightly as the elongate medical device bends or curves, to prevent wrinkling, buckling or kinking of the tube and external working channels. The tube and external working channels may be attached to the outer surface of the elongate medical device by a mechanical attachment (e.g., a tie, a hook, an anchor, etc.), a chemical attachment (e.g., an adhesive, etc.) or any other appropriate attachment.

In some examples, the tube having external working channels may be coupled to the elongate medical device in a manner that prevents prevent relative rotation of the external working channels relative to the elongate medical device. For example, the external working channels may be sufficiently elastic to minimize or prevent significant movement between the two, rotational or otherwise. Thus, in some examples the tube having the elastic (and external) working channels may move with the elongate medical device in a 1:1 ratio.

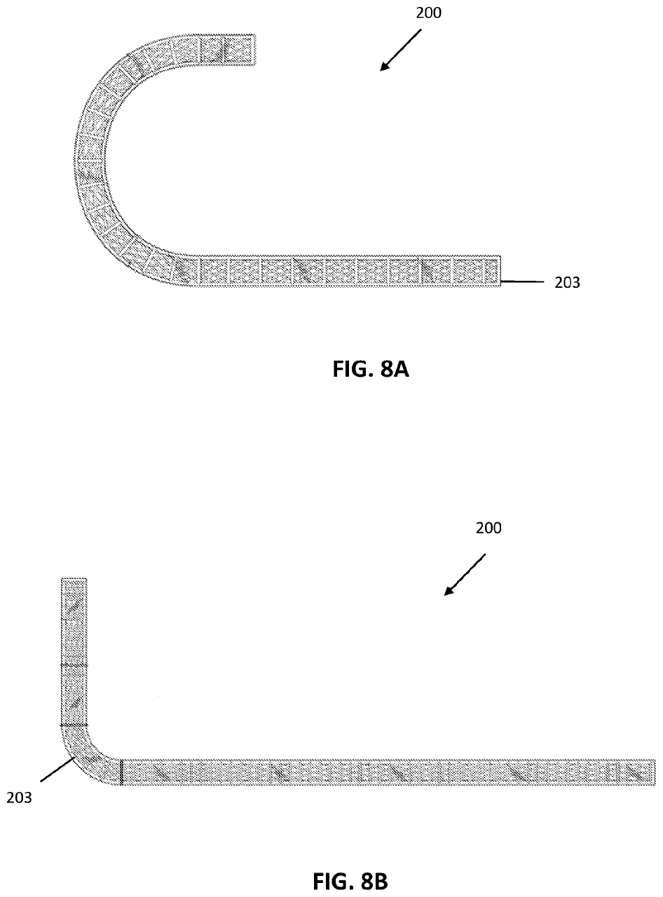

The engagement of the tube including working channels with the elongate medical device may be sufficient to minimize gaps between an interior surface of the tube 135 of the external channel device and the exterior surface of the elongate medical device (e.g., rigidizing device 200). The system 100 can include a flexible material, such as a with a weave pattern and/or filament(s) having an elastic core and a lubricious exterior. The elastic filaments forming the external working channels and/or tube may facilitate a snug and substantially close communication with the elongate medical device. As show in FIGS. 8A and 8B, an elongate medical device including a tube with one or more external working channels may operate when bent or curved through even highly tortious cures. FIG. 8A illustrates a curve of arc in a segment of the elongate medical device 200. Similarly, FIG. 8B illustrates a sharper bend in a segment of the elongate medical device 200. A tube with external working channels as described herein may be used with either configuration and even more tortuous bends without kinking or blocking passage through the external working channels. As shown in FIGS. 8A and 8B the tube and/or the external working channels may conform to these bends and curves (e.g., between 0 and 180 degrees or more, with a radius of curvature of approximately 1.5× the diameter of the elongate medical device without kinking, wrinkling, or the like. FIG. 8A shows a relatively large radius of curvature, while FIG. 8B shows a very narrow radius of curvature. The tube with external working channels described herein may track both of these curves. In general, where a segment or length of an elongate medical device 200 is curved or bent, the elasticity of the tube external working channels may be sufficient to maintain smooth and consistent contact with the rigidizing device 200 along an entire length which they are in communication with one another. The channels of the external working channels may possess similar capabilities as the tube 135 of the external channel device, such that the channels also continue a smooth exterior surface of the external channel device and rigidizing device assembly.

In general, a rigidizing devices can toggle between rigid and flexible configurations, with any number of transition cycles. As interventional medical devices are made longer and inserted deeper into the human body, and as they are expected to do more exacting therapeutic procedures, there is an increased need for precision and control. Selectively rigidizing devices as described herein can advantageously provide both the benefits of flexibility (when needed) and the benefits of stiffness (when needed). Further, the rigidizing devices described herein can be used, for example, with classic endoscopes, colonoscopes, overtubes, catheters, robotic systems, and/or navigation systems, such as those described in International Patent Application No. PCT/US2016/050290, filed Sep. 2, 2016, titled “DEVICE FOR ENDOSCOPIC ADVANCEMENT THROUGH THE SMALL INTESTINE,” the entirety of which is incorporated by referenced herein. The selectable rigid and flexible configurations of the rigidizing device may be accommodated by the tube with external working channels as described herein.

The elongate medical devices described herein can additionally or alternatively include any of the features described with respect to International Patent Application No. PCT/US2016/050290, filed on Sep. 2, 2016, titled “DEVICE FOR ENDOSCOPIC ADVANCEMENT THROUGH THE SMALL INTESTINE,” published as WO 2017/041052, International Patent Application No. PCT/US2018/042946, filed on Jul. 19, 2018, titled “DYNAMICALLY RIGIDIZING OVERTUBE,” published as WO 2019/018682, International Patent Application No. PCT/US2019/042650, filed on Jul. 19, 2019, titled “DYNAMICALLY RIGIDIZING COMPOSITE MEDICAL STRUCTURES,” published as WO 2020/018934, International Patent Application No. PCT/US2020/013937 filed on Jan. 16, 2020, titled “DYNAMICALLY RIGIDIZING COMPOSITE MEDICAL STRUCTURES,” and PCT/US2021/034292, filed on May 26, 2021, entitled “RIGIDIZING DEVICES” the entireties of which are incorporated by reference herein.

In some examples, the external working channels may be configured to allow the advancement of instruments or tools (i.e., working tools), such as surgical or laparoscopic tools, graspers, articulating graspers, fecal wash devices, and/or fecal-suctioning devices therethrough. In some examples, the tool can be a scope (e.g., so as to enable a secondary scope within or alongside a primary scope). The external working channel(s) can allow a tool to be guided along the elongate medical device until it reaches the distal end of the elongate medical device to perform the desired procedure. In some examples, the external working channels can accept a tool therethrough. For example, the elongate medical device may be coupled to a system 100 including one or more external and external working channels.

In some examples the proximal end of the tube with external working channels may be configured to engage with the channels formed by the external working channels. FIGS. 9A-9C show expanded views of a proximal end of an elongate medical device onto which a tube with external working channels 305 has been coupled. The elongate medical device in this example is a rigidizing device. The rigidizing device outer tube can be a thin-walled sleeve, such as an elastomeric sleeve, a plastic sleeve, or a cloth sleeve. In some examples, the rigidizing device outer tube can be a sleeve that is fiber reinforced or wire reinforced. In one specific example, the rigidizing device outer tube can be a cloth material that inherently has some stretch and/or a cloth material that is sewn at a 45° angle (e.g., such that being off-bias provides compliance and/or stretch). In some examples, the tube with external working channels can be permanently attached to (e.g., bonded, heat welded, sewn, or ultrasonically welded) the outer surface of the elongate medical device (e.g., rigidizing device 200). In any of these apparatuses the elastic working channels may be arranged linearly down the length of the elongate medical device at rest; e.g., the working channels may be straight (i.e., axially aligned with the rigidizing device outer tube 335). in some examples, the elastic working channels 305 may be spiraled. For example, the elastic working channels may be co-joined (to each other or to the elastic body) by sewing, bonding, or heat-sealing.

In some examples the elastic working channels may be lined with a hydrophilic, hydrophobic, or low friction (e.g., PTFE) coating. A liner insert tube 300 can include inserts (e.g., molded or extruded inserts) that are configured to be positioned within the external working channels 305 for use. For example, the liner insert tube 300 can be configured to be inserted into one or more external working channels 305 after a rigidizing elongate medical device 200 has been placed and/or rigidized in the body lumen. Each liner insert tube 300 can include a lumen 310 therein (configured for passage of a tool). Each liner insert tube 300 can have a stiffness sufficient to open or expand the external working channel 305 as it extends therethrough. In some examples the liner insert tube can have an inner diameter of between about 1 mm-7 mm, such as 3 mm-5 mm and a wall thickness of, e.g., between about 0.1 mm to 1 mm. The liner insert tube 300 can be made of a polymer, such as Teflon, FEP and/or a polyethylene (such as HDPE or LDPE). The lumen 310 can be lubricious to help enable passage of the tool therethrough. For example, the lumen 310 can be made of a material (e.g., the same material as the liner insert tube 300 itself) having a low coefficient of friction. As another example, the liner insert tube can be a composite structure that is coated with a separate lubricious coating, such as a hydrophilic coating.

As shown in FIGS. 9D and 9E, in some examples, the liner insert tube 300, which may equivalently be referred to herein as a guide, can include one or more projections (e.g., wings) and/or may have a symmetric or an asymmetric cross-section. For example, in some examples the apparatus may include one or more wings 315 that may have an asymmetric cross-section. In some examples the liner insert tube may include a non-circular shape with wings 315 (e.g., rounded triangular-shaped wings) extending from the region around the central lumen. In some examples, the wings 315 can form an angled surface 330 (e.g., having an angle, e.g., of 110° to 130°, such as approximately 120°) configured to conform closely to the outer circumference of the rigidizing device 200. The asymmetric cross section can advantageously ensure proper radial alignment of the liner insert tube 300 (e.g., so that the distal end of the lumen 310 points radially inwards with respect to the rigidizing device 200). Additionally, the asymmetric cross section can advantageously prevent rotational movement of the liner insert tube 300 within the external working channels 305. This can be particularly advantageous when the elongate medical device is a rigidizing device that is in a rigid configuration, as the asymmetric cross-section can help provide rigid and stable access to the desired working area. In other examples, the liner insert tube 300 can be symmetric and/or otherwise configured to be rotatable within the expandible working channel 305. In some examples, the proximal end of the liner insert tube 300 may include an indicator mark configured to indicate the rotational position of the distal end of the liner insert tube 300 relative to the working channel 305 and/or relative to the elongate medical device 200.

In FIGS. 9A-9B, the elongate medical device outer tube 335 can include a proximal manifold 320 attached thereto that may be configured to enable insertion of the liner insert tube 300 therein. For example, the manifold 320 can include ports 325 enabling access to each external working channel 305. In some examples, each port 325 can include a corresponding marker configured to enable identification of the working channel 305 (and thus identification of the distal circumferential position of a tool inserted therethrough). The markers can be shapes, numbers, colors, or any one of a wide variety of input/output matching identifiers. In some examples, the ports 325 can include a valve thereon and/or can be vacuum-sealed.

In some examples, the liner insert tube 300 can include a handle or stop 201 (see FIG. 9B) at the proximal end thereof to limit axial movement of the liner insert tube 300 too far within the working channel 305 and/or the manifold 320 that is coupled to the outside of the elongate medical device 335 (e.g., a rigidizing device outer tube).

FIG. 9C shows an example of a liner insert tube 300 (which may also be referred to herein as a guide) including a lumen 310 and a pair of wings 315; the liner insert tube is inserted into a port 325 that couples with the external and expandable working channels (not visible if FIG. 9C). Any of these apparatuses may include a port at the proximal end of the apparatus to assist in providing entry into the flat (e.g., unexpanded) working channel.

FIGS. 10A-10C illustrate another example of a liner insert tube 1000 as described herein. The liner insert tube may have a length that is approximately the same as (or just slightly longer than) that of the elongate medical device and external channels with which it may be used. The liner insert tube may exhibit high radial and axial stiffness while exhibiting low bending stiffness so that it may track through the external channel. FIG. 10B shows an enlarged view of the proximal end region 1033 and distal end region 1035. The proximal end region 1033 and distal end regions 1035 of the liner insert tube are shown. The proximal end 1033 may include an engagement 1037 that may engage or couple with a connection on the proximal end of either the elongate medical device and/or the tube forming the external channels.

The distal end region 1035 in this example includes a deflector 1041 that may be oriented so as to deflect a medical tool inserted through the elongate channel of the liner insert tube 1000 towards a midline of the elongate medical device or an endoscope inserted through the elongate medical device. For example, the distal tip also may be shaped to deflect a tool which passes out through the distal tip towards the center line of the elongate medical device (e.g., endoscope). This is illustrated in FIG. 10C, showing a deflector 1041 on the liner insert tool, which is designed to deflect a tool 1045 which passes through the liner inert tube 1000 towards the axial centerline of the elongate medical device (e.g., endoscope, not shown).

Both the proximal and distal ends of the liner insert tube 1000 in FIGS. 10A-10C include a pair of wings 1039, 1039′ as described above; the wings may guide and/or secure liner insert tube 1000 within the expandable channel. These wings (or “fins”) may help keep the liner insert tube 1000 aligned rotationally in the working channel. The fins may help to engage distal geometry so that, once fully advanced, it is ‘docked’, so as to prevent axial, lateral, and torsional motion.

FIGS. 11A-11C illustrate an example of a system as described herein, including a

tube 1101 formed of a knitted material that is attached over elongate medical device 1103 (in this example, a rigidizing overtube). The tube 1101 includes at least one expandable external channel 1105 into which a liner insert tube 1104 is inserted and coupled at the proximal end 1133 to the handle of the elongate medical device 1103. The liner insert tube may therefore hold the expandable external channel open and prevent snagging by a medical instrument or tool 1145 as it is passed through the liner insert tube 1100 to exit out of the distal end as shown in FIG. 11C. FIG. 11B shows the distal end of the liner insert tube 1104 including a deflector 1141 that directs the medical tool 1145 towards a midline of the elongate medical device 1103 or in any desired direction.

FIG. 11D shows an example of a distal end of a liner insert tube 1104 that is adjacent to an elongate medical device 1103, in this example, configured as an overtube 1103. The liner insert tube also include a deflector 1141 that is formed or attached to a distal end region of the liner insert tube 1104 and has a slight bend or angle at a distal region that directs a tool either distally (straight) or slightly radially inward but may prevent the tool from exiting radially outwards. In some examples the director may be rotated intentionally to direct a tool radially outwards rather than inwards.

In use, a working channel sleeve apparatus as described herein may include or may be part of an elongate medical device, including but not limited to a rigidizing device, for example, the working channel sleeve apparatus may be positioned or attached over an outer surface of the elongate medical device. The working channel sleeve apparatus, including the elongate medical device, may then be positioned at a desired anatomical location. If a user would like to deploy a tool at the anatomical position, a user can select an external working channel (e.g., based upon a marker, such as a color, symbol and/or text marker) at a proximal end of the elongate medical device, and the user can then insert either the tool or a liner insert tube 1104 through a port and into the external working channel. Inserting the liner insert tube (also referred to herein as a “guide”) can expand the external working channel. A tool can then be inserted through the liner insert tube. After the procedure is complete, the tool and liner insert tube can be removed, and the external working channel can collapse back down.

The liner insert tube can come in different sizes (e.g., with different sized lumens, such as lumens 1110 that range from 1 mm-7 mm, such as 2 mm-6 mm in diameter) and can be interchangeably used in external working channel(s). In some examples, the liner insert tubes can have a lumen without a bend at the distal end. In other examples, the liner insert tube may include a bend and/or asymmetric jog or turn configured so as to direct a tool inserted into the lumen of the liner insert tube in a desired direction, such as towards the center of the elongate medical device or away from the midline of the elongate medical device (e.g., so as to direct the tool radially outwards, such as for performing a procedure on a wall of the lumen). As described above, in some examples the liner inset tube includes a deflector 1141.

FIGS. 11E-11L illustrate an example of a liner insert tube 1104 that includes an inner lumen 1110 with a distal bend that steers a tool within the liner insert tube in a predefined direction (e.g., the liner insert tube may be configured to steer the tool towards a midline of the elongate medical device and/or may be oriented in the opposite direction, away from the midline of the elongate medical device. In any of the liner insert tubes described herein, the distal end of the liner insert tube may have an atraumatic and/or soft distal end 1142 configured to extend distally beyond the working channel (as shown in FIGS. 11J-11L). Further, as shown in FIGS. 11F-11I, the lumen 1110 can extend substantially axially within the liner insert tube 1104, but can be curved or slanted, e.g., radially inwards (e.g., at a 30°-60°, such as a 45° angle), just proximal to the soft distal end 1142 so as to direct the tool 1177 in a desired direction, such as towards the center of the elongate medical device. In some examples the liner insert tube 1104 can be steerable (e.g., via pull wires or other steering mechanisms) so as to enable further manipulation or directing of working tools passed therethrough.

The external working channels of the working channel sleeve apparatus may include one or more cuffs (e.g., elastic-like cuffs) or sections that may be configured to keep the external working channels collapsed against the elongate medical device when a tool or liner insert tube is not inserted therein.

FIGS. 11H-11L illustrate a liner insert tube 1104 having a lumen 1110 into which a tool 1177 may be inserted proximally so that it may extend distally from the elongate medical device 1103 at a distal end region, after passing through the external working channel 1181 (a “flat” or unexpanded external working channel 1181′ is radially offset from the first external working channel in which a liner insert tube has been inserted. FIGS. 11H-11L also illustrate a liner insert tube 1104 including wings 1115 that may maintain the orientation of the apparatus within the external and expandable working channel 1181. A tool 1177 is shown extended through the example liner insert tube shown in FIGS. 11D-11E.

As mentioned above, in some examples any of the tubes and/or the external channels formed on or as part of the tube may be formed of a knitted, woven or braided material. In some examples a knit fiber may be used to form the tube and channel(s). For example, FIG. 12A shows one example of knit material forming the tube and working channels described herein. FIG. 12A shows a magnified view of knit fibers 1255 forming a knit sleeve (tube) that includes four external channels. This knit pattern includes cross-connected rows of knit filament bundles. The precise knit pattern may be selected to optimize the expansion and other properties.

For example, FIG. 12B shows a portion of a template pattern for a knitted working channel sleeve 1200 including an inner knitted tube that is concurrently knit with external channels. In FIG. 12B, a front side of a knitted working channel sleeve is show flattened (it may be expanded open to form the tube); the back of the flattened tube portion is not visible. In this example, the bottom region 1203 shows an end of the knitted working channel sleeve before the external working channels 1220, 1220′ are begin; this region forms the inner tube of the working channel sleeve. Two external working channels 1220, 1220′ are shown on this side of the (flattened) working channel sleeve; these external working channels are knitted together and integrated with the inner tubular body. Each knit external working channel 1220, 1220′ include a proximal opening 1232, 1232′ on the end of the knit working channel.

The template pattern, including the stitch pattern shown in FIG. 12B may be generated and/or used by a knitting machine, such a flat (e.g., “flatbed”) knitting machine. In the example shown in FIG. 12B the knit forming the external working channels 1220, 1220′ may have a different pattern as compared to that of the inner tube portion 1203, 1228. For example, the knitted pattern for the working channels may be more elastic than the knitted pattern for the working channels. In some examples the knitted pattern for the working channels may have larger pores or higher porosity than that of the kitted pattern forming for the inner tube. In some examples the fiber(s) forming the knitted pattern for the external working channels may be different from the fiber(s) forming the knitted patter for the inner tube portion. For example, the fiber(s) forming the inner tube 1203 may be formed of a hybrid elastic and lower-friction material, such a hybrid a polytetrafluoroethylene (e.g., ePTFE)/spandex material, whereas the fiber(s) forming the external channels may be a low-friction material alone (e.g., Polytetrafluoroethylene, such as ePTFE), or an elastic material alone (e.g., Spandex).

As mentioned above, any of these working channel sleeve apparatuses may be formed by weft knitting, or warp knitting, including but not limited to tricot, Milanese knit, Raschel knit, and stitch-bonding. Any appropriate size filament(s) may be used. For example in some examples the filament(s) (e.g., fiber or fibers) forming the outer working channel(s) may be about 100 denier to 6000 denier for the outer wrap. Fiber(s) for the inner elastic core could be 400 to 6000 denier for the inner wrap.

In some examples the external channels using this kind of a knitting machine (e.g., a flatbed knitting machine) may have the advantage of being made complete in one piece without any post knitting assembly or sewing required. A knit structure also lends itself to creating stretchy fabrics, more so than with woven constructions.

In some examples a higher gauge knitting machine would be advantageous so that the density of the knit could be made tighter and smoother, reducing sliding friction and making the fabric more puncture resistant. In FIG. 12A, the knit was formed using between 1-4 filament ends, having fibers of Denier (thickness) of between about 10-200. The knitting machine gauge may be between about 10/14 and 16/18. In the example shown in FIG. 12A, the fiber used for knitting may be, e.g., Polypropylene, PTFE, UHMWPE (Ultra High Molecular Weight Polyethylene, otherwise known as Spectra or Dyneema), silicone, polyurethane, etc. As described above, the fiber may be a hybrid elastic/lubricious (e.g., elastic core wrapped with a lubricious material).

Alternatively or additionally a woven material may be used to form the external working channels.

In use, an external working channel device (e.g., a tube having one or more external working channels) may be installed over an elongate medical device (e.g., a rigidizing device). The main tube lumen of the external working channel device 100 may sufficiently expand against the elastic force of the to receive the elongate medical device 200. The assembly including the elongate medical device and external working channel(s) may then be inserted into a body (e.g., a body lumen, a body vessel, etc.) through a natural or artificial orifice and advanced according to the need of the procedure being performed. When the distal end of the assembly is positioned in the desired location within the body, a working channel of the external working channel device 100 may be selected to receive a liner insert tube or tool directly therein. The tool or liner insert tube may be advanced through the working channel, expanding it. In examples in which the elongate medical device is rigidizable, the liner insert tube or tool may be inserted after the elongate medical device has been rigidized to retain a shape and define a path of the external working channel through which the liner insert tube or tool may be passed through. The distal end of the liner insert tube or tool may be advanced to a desired location and the procedure may continue through operation of the tool distal end.

For example, FIG. 13 illustrates a method of positioning a tool within a body. In this example the method may include inserting an elongate medical device into the body, where the elongate medical body includes a tube forming one or more external working channels 1301. In some examples the tube is attached so that it may slide or move slightly relative to the outer surface of the elongate medical device. In examples in which the elongate medical device is configured as a rigidizing member, the rigidizing member may initially be in a flexible configuration. The elongate distal end of the elongate medical device may be positioned at or near a target region of the body to be treated 1303. A liner insert tube and/or a tool may be inserted into an expandible working channel, so that the external working channel expands to accommodate the liner insert tube 1305. In some examples the liner insert tube may be engaged with a distal and/or proximal end of the tube and/or the elongate medical device. A working tool may be inserted through the liner insert tube and out of a distal end of the liner insert tube 1307. Thereafter, a procedure may be performed on the target region using the working tool 1309. Additionally, rigidization could occur, for example, between steps 1303 and 1305 (i.e., 1304)

### EXAMPLES

FIG. 14A-14C illustrate one example of a system 1400 including an elongate medical device 1403 (e.g., an overtube into which an endoscope has been inserted in FIGS. 14B and 14C) including a tube 1401 forming a plurality of external and expandable working channels. In FIG. 14A the elongate medical device is shown with the tube attached over the outer surface of the elongate medical device, and a liner insert tube 1404 extends through one of the expandable channels and out of the distal end. As mentioned, the elongate medical tool is configured as a rigidizing overtube over which a tube or sleeve 1401 including a plurality of external working channels have been integrated. The proximal handle 1406 of the rigidizing endoscope also includes access inlet into each of the external working channels. These working channels may be marked (e.g., by a symbol, code, alphanumeric, etc.) so that the user may know where around the radius of the distal end the medical tool will exit the working channel.

FIG. 14B shows an example of both an external working channel 1405 on the rigidizing tube shown in FIG. 14A, with a tool 1407 inserted at proximal end of the endoscope and emerging from distal end, as shown in FIG. 14B. In this example an endoscope 1413 is also included inserted through the overtube, and may itself include a working channel, configured as a traditional internal working channel. In FIG. 14A a second medical tool 1407′ is inserted through this internal (endoscope) working channel, as shown.

FIG. 14C illustrate an example of a medical tool 1407′″ inserted through a liner insert tube 1404 that is itself inserted through the expandable external working channel 1405. A blunt ended deflector 1434 on the liner insert tube 1404 is shown deflecting the tool radially inward relative to the overtube and endoscope.

Any of the apparatuses described herein may be used with a robotic system, including a robotic endoscope system. FIGS. 15A and 15B illustrate operation of a system that may include a pair of telescoping devices, including an internal endoscope 1545 (“child”) and an external overtube (“mother”) over which may be worn an inner tube 1547 including four external working channels 1504. The tubes, such as the knitted, woven and/or braided tubes described above, forming the external working channels may be kept in a collapsed configuration when navigating the apparatus through the body lumen. Once at or near a target region, the external working channels may expand or allowed to expand as shown in FIG. 15B. In this example FIG. 15A shows the apparatus with the external working channels 1504 in a collapsed configuration.

FIG. 15C shows an example of a distal end region of the endoscope and overtube with external working channels 1504 attached as part of a sleeve or tube on an outside of the overtube. In this example three tools 1507 are shown extending and may manipulate tissue as described herein.

FIGS. 16A-16D illustrate another example of a system 1600 including an outer elongate medical device 1646 onto which a plurality of external working channels 1644, 1644′, 1644″ have been formed as part of a tube or sleeve 1603 (e.g., working channel sleeve). An inner medical device 1645 (e.g., endoscope) is shown inserted through the outer medical device. The tube or sleeve 1603 is coupled distally to the distal end region of the medical device 1646 and the proximal end is coupled to a proximal end region of the medical device including an introducer region 1616 that provides access into each of the collapsed or partially collapsed external working channels (shown as color coded in this example). In examples including an endoscope 1645 that may visualize the distal end region of the apparatus, the endoscope 1645 may detect when the tool or a liner insert tube 1604 exits by correlating which color and/or marking 1688 indicates where the tool 1607 was inserted or introduced into the working channel. FIG. 16C shows another example of the same system as in FIGS. 16A-16B but with a second tool 1607′ inserted into a second channel of the elongate tube. FIG. 16D shows an enlarged view of the distal end region of the robotic system including an outer elongate medical device 1646 (configured as a mother or overtube), an inner elongate medical device 1645 (configured as a child, shown as an endoscope) and a tool 1607, configured as a suction tube. In some examples a liner insert tool may be used (not shown).

FIG. 17 illustrates the use of an expandable external channel 1745 as described above on a rigidizing overtube. In this example a tool 1707 inserted into an external channel 1745 colored orange at the proximal end emerging from the external channel at the distal end, which is also colored (e.g., orange) for identification. For example, FIG. 18 is an example showing the view of the color-coded distal tip from the perspective of an endoscope's camera, showing four color-coded regions 1888, 1888′, 1888″, 1888′″ indicating where the external channels are located (and where a tool will exit) relative to the patient.

As mentioned above, existing external working channel apparatuses are particularly ill-suited for use with long are more flexible elongate members (e.g., catheters, endoscopes, overtube, etc.). Such systems may not address the issues of maintaining flexibility of the flexibility elongate member, and/or of binding-up or wrinkling of the external working channel apparatus on the elongate flexible member. In particular, existing external working channel apparatuses, particularly those made of an elastic material, may lead to loop catching and “pocketing” or pouching of the external working channel as a tool is inserted through the working channel. Merely creating a channel does not inherently create the proper conditions for a long length tool to pass within over a high tortuous path. For example, FIGS. 19A-19C illustrate problems that may arise when inserting and/or passing a tool through an external working channel, and particularly those formed of an elastic material, such as a silicone, latex, or spandex material, which may result in the previously mentioned drag issues.

For example, FIG. 19A illustrates one example of an external working channel apparatus 1904 that is applied over an elongate member 1902, shown schematically in cross-section. In FIG. 19A, a tool 1906 is inserted into the working channel 1905 formed in the external working channel apparatus. As the tool 1906 is moved forward 1910 (as shown in FIG. 19B), the external working channel apparatus may constrict movement, resulting in pocketing 1908, which may be particularly problematic when the working channel of the external working channel apparatus is formed of an elastic material and/or where the underlying elongate member 1902 and the applied external working channel apparatus 1904 is bending or curved. Once a tool has pocketed, the application of additional force only serves to buckle the tool shaft while the tip pushes further into material from which it would not advance.

FIG. 19C illustrates the issue of the increasing drag due to the normal force of the working channel when a tool 1906 is inserted into and through the working channel 1905. As shown, even in instances where the tool 1906 is inserted without catching, pocketing or pouching (as shown in FIGS. 19A-19B), the external working channel 1904 may be held against the tool both along the length of the tool within the working channel, as well as the tip end of the tool, resulting in a normal force 1914 (again, applied at both the tip and along the shaft length), both of which result in drag on the tool. This problem is particularly acute when the working channel is formed of an elastic material; further, the longer the channel and the higher the associated tortuosity, the larger the drag force, which may make it extremely difficult (or even impossible) to insert the tool through the working channel and may lead to jamming or even buckling of the tool within the working channel.

FIG. 19D illustrates an example of an external working channel apparatus 1904 (e.g., “working channel sleeve apparatus”) that is applied over an elongate member (e.g., an overtube), in which the endoscope is curving, and the tool has caused the working channel to pocket or pouch, preventing it from advancing any further. In FIG. 19D, the tool 1906′ (covered by the material of the external working channel) has been advanced through the external working channel 1905 and has formed a pouch 1934 in the material of the external working channel. In this example the external working channel is a knit formed of an elastic filament (e.g., a spandex material). The knit in this example is vertical (e.g., not horizontal).

In general, any of the external working channel apparatuses described herein may include one or more of features configured to address these problems and provide additional advantages and benefits. The external working channel apparatuses described herein may be particularly well suited for flexible and longer elongate members (e.g., endoscopes, catheters, etc.), and may prevent or reduce the issues discussed above, including those illustrated in FIGS. 19A-19D. For example, the external working channel apparatuses described herein may be configured to prevent reducing the flexibility of the elongate member onto which it is applied, and may be configured to prevent or reduce bunching or binding-up, even when inserted through regions of a body that apply radial inward force. In general, these apparatuses may include one or more features that reduce friction, normal forces and capstan drag when inserting and manipulating tools within the external working channels. The features describe herein may be used individually or in combination and may control the friction between the working channels and the tools inserted through them (e.g., reducing the normal force when using the working channels) and/or between the elongate member and the core of the external working channels apparatus.

### Siding, Non-Elastic Filaments

Any of these apparatuses described herein (e.g., devices, systems, assemblies, etc.) or methods of using or making them may include the use of one or more non-elastic filaments. For example, either the one or more working channels and/or the core region may be formed of one or more filaments of a nonelastic material. These non-elastic filaments may be configured to slide relative to each other; this sliding movement may allow the working channel to mechanically expand and/or contract without stretching the individual filaments. As described herein, the one or more non-elastic filaments may form pores or opening through the outer working channels that can change dimensions (getting bigger/smaller) as the assembly bends and/or as one or more tool is inserted into and/or through the external working channel. Thus, these filaments may be allowed to slide, but may not significantly stretch. The filaments may slide or shear relative to each other to allow the working channel (and in some cases the core region) to expand and contract, e.g., providing a “mechanical stretch,” and thereby change the overall shape of the assembly without deforming the filaments. This may be helpful because it may prevent snagging.

Examples of appropriate non-elastic material may include “plastic” materials such as, but not limited to: PTFE, Polyester, UHMWPE, HDPE, polypropylene (in contrast to Spandex/Elastane/Latex and Silicone, which may be considered “elastic” materials). For example, PET or HDPE may be used. The use of these plastic materials may also allow thermal, ultrasonic, and/or adhesive bonding. In some examples appropriate non-elastic materials may have a modulus of elasticity of 20,000 psi (e.g., 20,000 psi or greater, 30,000 psi or greater, 40,000 psi or greater, 60,000 psi or greater, 100,000 psi or greater, 200,000 psi or greater, 500,000 psi or greater, 750,000 psi or greater, etc.). The non-elastic materials that may be used may typically have a lower coefficient of friction (e.g., 0.6 or less, 0.55 or less, 0.5 or less, 0.45 or less, 0.4 or less, etc.) compared to more elastic materials. As described above, the use of these non-elastic materials, may prevent tools from catching or snagging (e.g., “pooching”). In any of these methods, thermal, ultrasonic welding, or adhesive bonding may be utilized instead of sewing.

Any of the apparatuses described herein may be configured so that they stretch anisotropically. In particular, any of the apparatuses described herein may be configured to have a higher stretch (e.g., mechanical stretch), particularly when using the non-elastic filaments described herein, in an axial direction, e.g., along the length of the apparatus, rather than in the perpendicular direction, i.e., radially. Thus, these apparatuses may have a high axial stretch and a low hoop stretch. This property may permit the apparatus to allow bending (e.g., flexibility) while limiting radial expansion, which may otherwise lead to snagging or pouching.

Any of the external working channel apparatuses described herein may include one or more hydrophilic (or hydrophobic) materials, and in particular lubricous coatings, particularly within the inside of the working channels. This may be done by the use of a coating, a liner, etc. For example, a hydrophilic coating may be used, including on an outer surface of the tools.

In general, it may be particularly beneficial to use a material for forming the external working channel assembly that has a similar wet/dry behavior. For example, the material forming the working channels and/or the core region may have similar wet/dry behavior, such that the percentage change of the behavior of the material (e.g., swelling, friction, etc.) between wet to dry may have a maximum percent change of 35% difference or less (e.g., 25% or less, 20% or less, 18% or less, 15% or less, 12% or less, 10% or less, 8% or less, 5% or less, etc.). Examples of materials having similar wet/dry behavior include, but are not limited to, PTFE. PTFE and many of the other non-elastic materials described herein have essentially the same surface friction (for sliding) when wet as when dry. Further, they do not swell or otherwise change dimensions significantly when wet as compared to dry. Materials with identical or comparable dry/wet properties may be particularly beneficial as the apparatuses described herein may be used in both dry and wet (often simultaneously) environments, as when inserted into the body.

In general, any of the working channel apparatuses described herein may be configured so that they are attached to the medical device (e.g., the flexible, elongate member, such as a catheter, endoscope, overtube, etc.) at the distal and proximal end regions but are relatively unattached to the region of the outer body of the medical device between the proximal and distal end regions. This configuration may help prevent the apparatus from limiting the ability of the underlying medical device to bend. However this may make it easier for the apparatus to bind up, particularly when inserting or positioning such an apparatus within a body lumen in a region in which radially inward pressure holds the external working channels or prevents the external working channel region from moving, which may lead to bunching or binding of the external working channel relative to the elongate flexible member (e.g., endoscope, catheter, overtube, etc.).

In any of the apparatuses described herein, this potential bunching or binding may be addressed by adjusting the friction between the core portion of the working channel and the endoscope. Any of these apparatuses (e.g., devices, systems, assemblies, etc.) may control the coefficient of friction between the core portion of the working channel assembly and the outer surface of the medical device over which the core portion extends. For example, the coefficient of friction between the outer surface of the elongate, flexible member and the inner surface of the core region may be between about 0.3 and 1 (e.g., between about 0.35 and 1, between about 0.4 and 1, between about 0.45 and 1, between about 0.5 and 1, between about 0.55 and 1, between about 0.6 and 1, etc.). Having a slightly larger coefficient of friction, as compared with the coefficient of friction between the inside of the working channel and the tool, may prevent the working channel assembly from bunching up on the medical device in use. The potential bunching or binding could also be modulated by localized attachments, intermittent attachments, regular attachment, episodic attachments, bonding, or shear feature integration.

In any of the apparatuses described herein, the apparatus may be configured to change the stretch (‘mechanical stretch’) along the length of the apparatus. For example, in any of these apparatuses the working channel apparatus may change its elasticity as it extends along the length of the medical device. In some examples the working channel assembly (e.g., the core region) may be more elastic (gripping) at the distal end and proximal end than in the middle region, which may allow it to be more flexible. Alternatively in some examples the apparatus may be configured to increase the flexible of the working channels along the length, so that the distal end region is more flexible that the more proximal regions.

As mentioned above the angle of the filaments forming the working channel assembly (e.g., the one or more working channels) may be arranged along the length of the working channels to reduce drag as a tool or tools is inserted into the working channel. In some examples the apparatus may be configured so that one or more axial tensioning members may help prevent catching/pouching. As described above in reference to FIGS. 19A-19C, one way that ‘pocketing’ issues occur is when an area of loose textiles hook under the bottom of the liner tool. As the liner tool continues forward the textile that is hooked under acts as a tensile member starts to restrict the tool movement as they are not able escape from underneath the tool. Any of the apparatuses described herein may be configured so that the resulting textiles (e.g., working channel region) is arranged on the bias of the filaments. For example, the filaments may be arranged in an approximately 45 degree interlacing pattern. This pattern may also or alternatively help with ramping over the liner tool and not getting caught the tool under the filaments forming the apparatus. Additionally, axial tensile members may be included, similar to the horizontal knit described below.

Alternatively or additionally, any of these apparatuses may be configured so that the filaments are biased on the braid to prevent/reduce catching of the tool on the filaments forming the working channel(s).

In general, the apparatuses described herein may include a plurality of pores or openings through the working channel portion of the apparatus. The size of pores may be optimized. In general, if these pores are too large, the tool may catch on the pores; if they are too small the working channel may not work effectively, as the resulting material may not have a sufficiently tight sliding distance.

Any of these apparatuses may be formed of a knit material, as described and illustrated above. In some cases the knit may be a horizontal knit, as shown in FIGS. 20A and 20B. The direction of the knit may be knitted horizontally. As mentioned, the use of a horizontal knit, particularly when using non-elastic filament, may help provide a desirable anisotropic stretch profile with a lower hoop stretch than an axial stretch. The anisotropic stretch profile may prevent pouching (“pooching”), pocketing, catching, snagging and the like, and may reduce drag. FIG. 20A show an external working channel apparatus 2000 that is formed by a horizontal knit 2034 to form four external working channels 2004 over the core region, which may be attached proximally and distally over the elongate, flexible member (e.g., catheter, etc.). In this example, the direction of the coursing of the knit is horizontal, e.g., in a plane perpendicular to the long axis of the elongate flexible member. The example shown in FIGS. 20A and 20B are also rib knots; the knit may be formed in opposite directions on a walewise basis. For example, FIG. 20B illustrates a portion of the length of the tube is knitted horizontally along the length (e.g., of the machine), so that ˜600 wales by ˜100 courses. Previous tubes were knitted with ˜100 wales×600 courses. The working (e.g., tool) channel may then pass along the technical back of the fabric in the direction in which it was knitted, along the courses. The tool channel slides easily in this direction. The knitting may be flat knitting or warp. In any of these examples the knitting may be performed so that the working channels, including the core region may be generated as a single tubular knit.

FIGS. 20C and 20D show examples of external working channel apparatuses similar to those shown in FIGS. 20A-20B, in which both the working channels (four are included in this example) and the core region are formed by a knitted material (in this case as a unitary knit body) that is formed as a horizontal knit using a non-elastic (PTFE) fiber. In FIGS. 20C-20D three of the external working channels have been expanded by inserting tubular forms 2064 to illustrate fully expanding the external working channels. The horizontal knit also forms the core region 2030 in this example, and the core region extends over the elongate member, shown as an overtube 2077 in FIGS. 20C and 20D.

As would be understood by those of reasonable skill in the art, knits have 2 faces—a technical face and a technical back. In some examples, the apparatus may be configured to orient the knit (technical fact/back) so that the inner lumen of the working channel(s) may configure to face the technical face (or the technical back). The technical front of the knit may have more ridges/projections, which may result in slightly greater friction. Thus, in some examples it may be beneficial to orient the technical front on the outside of the working channels and/or on the inner surface of the core region.

FIG. 21 shows an example of a material (shown here as a knit material) that is configured to include openings (e.g. pores) formed by the knitting process using a non-elastic knitting material. In this example, the pores may change shape during use, allowing the individual strands of filament to slide or shear relative to each other, which may enlarge and shrink the sizes of these opening. In FIG. 21 the pores have a relative length of about 1.4 mm (e.g., 1.487, 1.410, 1.413) and a relative width of about 0.55 (e.g.,0.575, 0.546, 0.576). in use, the application.

Any of the apparatuses described herein may include a braided structure, such as that shown in FIGS. 22A-22B, showing an external working channel assembly formed as a multi-lumen braid. In FIG. 22A the braid is shown forming over a first mandrel 2216, holding the shape of the medical device to form the core region 2220, and a second mandrel 2218, forming the external working channel pattern 2222. Although the braiding pattern shown in FIGS. 22A-22B illustrate just one working channel, this configuration may be modified to include multiple external working channels. In FIGS. 22A-22B, the filaments (e.g., yarns) that make up the external working channels may be brought out of the main core braid and then rejoined with the core braid to enable movement of the yarns and a cohesive structure.

In general, the external working channel apparatuses described herein may be formed as a unitary structure, in which the core region that is applied over the elongate member (e.g., endoscope, overtube, catheter, etc.) may be integrally formed with the one or more working channels. Thus, the different regions may be non-separable. For example a knitted, woven and/or braided structure (as shown in FIG. 22A) may include both the core and working channel(s) formed together, e.g., sharing one or more filaments.

The working channels described herein may include one or more axial tensioning members, particularly for variations that are braided, which may help with keeping the material forming the braid from catching or snagging a tool being inserted. This is illustrated in FIGS. 23A-23B, in which an external working channel 2304 extends over a flexible elongate inner member 2306. A liner tool 2307 is shown extending through the braids. As the liner tool is moved forward, the tensile members in external working channels may constrict movement. The braid biased construction may help act as a ramp to keep textile from catching under the liner tool.

FIGS. 24A-24B illustrate another example of a braid, configured as a double layered braid, that may be used with any of them methods and apparatuses described herein. In this example, a double layered braid includes two braids, one core and a second one that forms the working channel. The outer layer may be compressed braid externally that is connected through some means (e.g., TPU reflow) along multiple axial lines to create working channels 2428. In FIG. 24 the working channel apparatus 2404, over core 2430. In FIGS. 24A-24B there may be multiple braids, e.g., each working channel is its own braid, and they may be connected at distal and proximal ends.

FIG. 25 illustrates another example of an apparatus including multiple (e.g., four are shown) channels formed of braids 2564 that are arranged over an elongate, flexible member 2566 either with or without (as shown) the core region adjacent to the elongate flexible member. In FIG. 25, the working channel braided regions only connected to the device at a proximal and distal end. An outer external elastomeric outer layer may be included to cover (and in some cases to lock down onto) the inner elongate working channels.

FIG. 26A illustrate another example of a working channel apparatus 2604 formed by sewing. An inner (core) layer may be stitched to an outer (external working channel) layer. The top layer may be sewn with stitches that are sufficiently robust and small to maintain the external working channel while avoiding snagging. In this example, a discrete layer is sewn or otherwise attached to a discrete layer, i.e. a layer that existed as a separate entity before it was co-joined. In the example of the knitted structure, there are no clear separable layers.

In some examples, the materials forming the core region and external working channels may be selected to allow heath processing (e.g., fusing), as illustrated in FIG. 26B. In this example, a core 2630 material, which may be any appropriate material or fabric having sufficient internal support to hold onto the medical device. The top layer may be thinner, and/or more flexible, such as a fabric that is resistant to wrinkling. In some examples these materials may be thermoplastic polyurethane materials.

Fabric-based apparatuses such as those shown in FIGS. 26A-26B may be particularly amenable to coating (e.g., with hydrophobic material to be made lubricious). In general, a different fabric may be used for core and for working channels. In some examples these materials may have different elasticities (e.g., a more elastic material for the core region and a less elastic material for the channels; alternatively the apparatus may include a less elastic material for the core, and a more elastic material for the channels).

In general, as shown in these figures, any of these devices (including knit, woven, braided, etc.) may include a different pore size for the core region versus the working channels (e.g., larger pores for core/smaller for elastic, smaller for core/larger for elastic).

Any of the apparatuses described herein may be adapted to include a distal tip region that is narrowed or shaped, including one or more guides or ramps, including regions that are narrowed relative to a more proximal regions, in order to help guide the direction out of the distal end of the working channel. For example, the distal tip of the apparatus may include a narrowing at the distal end to help guide the direction out of the distal end. Any of these apparatuses may include a deflector at the distal end (e.g., to deflect away from, or optionally towards, the midline). These apparatuses may also be configured to provide a sense of tactile feedback as the tool or tools exit the working channels.

### Tools

Also described herein are tools configured to be used with the working channels described herein. In particular, described herein are sets of tools (e.g., matched sets) that may go together through any of the working channels described herein. In general, one of these tools may be used for steering/guiding the other tool. This is illustrated in FIGS. 27A-27D. These tools may be designed as a matched set to the working channels, such that they have optimized performance when used together, operating in a way that would not achieve such results should non-set-matched tools be utilized.

In FIG. 27A the working channel includes a deflector 2738 that deflect the tool out and radially away from the midline of the elongate member 2766 (endoscope). The apparatus also includes a working channel 2728 coupled to a core region 2730 that is held over the elongate member. A pair of matched tools 2754, 2750 is extending out of the distal end of the working channel. In this example, the tools include a steerable insert liner tool 2754 that has a steerable distal end. The second tool in this example is a grasper tool 2750 that may fit through a lumen in the steerable insert liner. FIG. 27B shows a view of the entire assembly of FIG. 27A, including the proximal and distal ends. The liner tool 2754 includes a handle 2741 with one or more controls for controlling steering of the liner tool. The proximal end of the grasper tool 2750 passes through the handle and the set of tools are inserted through the working channel 2728 from the working channel interface 2716 at the proximal end of the flexible elongate member 2769. In this example, the proximal elongate member is configured as a rigidizing endoscope that also includes a flush port/line 2739 and a pressure port/line 2737 for rigidizing the device. FIGS. 27C and 27D show enlarged view of the distal end of the assembly 2740, showing rotational steering of the steerable liner tool 2754 extending from the distal end of the elongate member 2766. The grasper tool 2750 is extending from the lumen of the liner tool 2754, and is rotatable 2755 relative to the elongate flexible member (e.g. endoscope).

FIGS. 28A-28C illustrate an example of a handle 2841 for a liner tube tool 2854 that may be matched with and may steer a second tool (e.g., grasper) 2850. The handle includes one or more controls 2846 to steer the liner and therefore the deflection and position of the second tool. A second variation of a handle is shown in FIGS. 28B-28C, including a plurality of controls 2846, 2846′ (e.g., for steering left/right), 2847 (e.g., for locking/unlocking the second tool within the first tool). The tools described herein may be mono-directional, bi-directional, or movable in

more than two directions (e.g., multiple orientation usage). The nature of the tool liner with the expanding working channels may allow it to torque the tool liner or an inner tool by torquing the handle at any angle. To enable easier usage, the controls can be designed to be accessible from either side. Any of these apparatus may include markers on the shaft for the liner tool (e.g., depth markers, etc.). For example, device marker bands can be included as part of the tool liner shaft to signify various positions (tip at distal end of working channel, optimal usage position, etc.). The tool liner may generally be torqueable. In general, the tool liners may be torqued for positioning of tools. Any of the tools described herein may include a shape memory/superelastic material, such as Nitinol or other wire form. This material may allow for traction without requiring a working channel allowing another tool to be put through. Any of these apparatuses ay include a concentric tube continuum/active cannula control. Rather than articulating, the device may be controlled with multiple concentric cubes of varying curves and stiffness to create positioning.

Other tools may include irrigation tools (FIGS. 29A-26C) and irrigation tools (FIGS. 30A-30E). In FIGS. 29A the suction catheter 2971 includes an elongate body having a distal end (tip) region 2973 and a proximal connector 2977 end. The tip 2973 may be configured as a radial suction tip (FIG. 29B) with a capped distal end 2977 having a plurality of lateral openings 2975 for suction. FIG. 29C shows a distal tip 2973′ with a distal opening 2977′ and lateral side openings. An irrigation device 3074 (FIG. 30A) may also include an elongate body with a distal tip region 3073 and proximal connector 3077. FIGS. 30B-30E illustrate different variations of distal tips 3083′ that may be used, including radial spray openings 3085 (FIG. 30B) and a closed distal tip 3087, distal shower openings 3085′ (FIG. 30C), 45 degree or 90 degree spay tips having angled distal spray openings 3085″, or direct spray tips (FIG. 30E) having a single distal opening 3085″. Other tips and other tools may be used as well.

### Robotic Apparatuses

As mentioned above, the external working channel apparatuses described herein may be configured as part of a robotic system or for use with robotic apparatuses. In some examples the external working channel apparatus may be attached (including removably attached) over an outer tubular member that is robotically controlled, such as a robotically controlled overtube and/or endoscope assembly. FIG. 16 shows an exemplary apparatus 3100, including an external working channel 3101 (according to any of the examples described herein) extending over an overtube 3112; the system may optionally include an inner endoscope 3110. The overtube and inner endoscope can be separately or collectively be robotically controlled or manipulated (e.g., steering, movement, rotation, etc. including in some examples, rigidizing). As shown in FIG. 31, the outer overtube 3100 and the inner endoscope 3110 may be terminated together into a common structure, such as a cassette 3157. The outer overtube 3100 can be movable with respect to the endoscope 3110 by rotation of a driver mounted to the cassette 3157. The system may include actuators 3171a, 3171b that may connect to cables 3163a,b respectively, to steer (e.g., bend or deflect) the tip of the endoscope 3110 (and/or outer overtube 3112). Other steering mechanisms (e.g., pneumatics, hydraulics, shape memory alloys, EAP (electro-active polymers), or motors) are also possible. The cassette 3157 can further include bellows 3103a, 3103b that may connect to the pressure gap of the endoscope 3110 and the overtube 3112, respectively to drive fluid through pressure lines 3105z, in variations for either the endoscope and/or the overtube that are configured to rigidize when pressure is applied. As shown in this example, the cassette 3157 can include eccentric cams 3174a,b to control bellows 3103a,b. Alternatively, one or more linear actuators can be configured to actuate the bellows. As another alternative, the devices can be rigidized and de-rigidized through one or more pumps or pressure sources (e.g., via pressure line 3105z).

The working channels may be configured to allow the use of one or more external working channels during operation of the apparatus. The insertion/removal into and out of the external working channels may be coordinated by the robotic system.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-21. (canceled)

22. An external working channel assembly, the assembly comprising:
an elongate, flexible member;
a core region extending over an outer surface of an elongate, flexible member, wherein the core region is coupled to the outer surface of the elongate flexible member at a distal end region or the elongate flexible member and a proximal end region of the elongate flexible member, but is not secured to the elongate flexible member between the distal end region and the proximal end region; and
an external working channel extending along an outer surface of the core region, wherein the external working channel is configured to accommodate a tool inserted through the external working channel,
further wherein a coefficient of friction between core region and the outer surface of the elongate flexible member is between 0.3 and 1 to prevent bunching of the core region and external working channel when operating the assembly.

23. The assembly of claim 22, wherein the coefficient of friction is between 0.4 and 1.

24. The assembly of claim 22, wherein the external working channel is formed of one or more non-elastic filaments.

25. The assembly of claim 24, wherein the external working channel comprises a plurality of pore openings formed by the one or more non-elastic filaments and configured to change dimension as the one or more non-elastic filaments slide over each other.

26. The assembly of claim 22, wherein the external working channel is knitted or braided.

27. The assembly of claim 24, wherein the one or more non-elastic filaments comprise a plastic material having a modulus of elasticity of greater than 50,000 psi.

28. The assembly of claim 24, wherein the one or more non-elastic filaments comprises one of: polytetrafluoroethylene (PTFE), polyester, ultra-high-molecular-weight polyethylene (UHMWPE), high density polyethylene (HDPE), and polypropylene.

29. The assembly of claim 24, wherein the one or more non-elastic filaments comprise a plastic material having a coefficient of friction of 0.5 or less.

30. The assembly of claim 22, wherein the core region is formed of one or more filaments.

31. The assembly of claim 24, wherein the core region is formed of at least some of the filaments of the one or more filaments forming the external working channel.

32. The assembly of claim 22, further comprising a plurality of external working channels extending along the outer surface of the core region.

33. The assembly of claim 22, wherein the external working channel is configured to have an anisotropic stretch profile with a lower hoop stretch than an axial stretch.

34. The assembly of claim 22, wherein the elongate, flexible member comprises an endoscope or an overtube.

35. The assembly of claim 22, wherein the elongate, flexible member comprises a rigidizing device.

36. The assembly of claim 22, wherein the core region and the external working channel comprises a multi-lumen braid.

37. The assembly of claim 22, wherein the core region and the external working channel comprises horizontal knit.

38. The assembly of claim 22, further comprising a matched pair of tools configured to be inserted together through the external working channel, wherein a first tool of the pair of tools comprises a steerable distal end configured to steer a second tool of the pair of distal tools as it exits the external working channel.

39. The assembly of claim 22, further wherein the external working channel comprises a lubricious coating.

40. The assembly of claim 22, further comprising a deflector at a distal end of the external working channel configured to defect a tool extending from a distal end of the external working channel radially away from the distal end of the elongate, flexible member.

41-.56. (canceled)

57. A system including an expandable external working channel, the system comprising:
an elongate medical device having a flexible or selectively rigidizable body;
a knit, woven or braided tube extending over an outer surface of the elongate medical device and formed of one or more non-elastic filaments; and
one or more knit, woven or braided external and expandable working channels integrally formed along a length of the tube and configured to receive a medical tool inserted therethrough, wherein the knit, woven or braided external and expandable working channels are formed of one or more non-elastic filaments.

58.-59. (canceled)

60. A method of positioning a tool within a body, the method comprising:
inserting an elongate medical device comprising a rigidizing member into a body while the rigidizing member is in a flexible configuration, so that a knit, braided or woven tube extending over an outer surface of the elongate medical device may slide relative to the outer surface;
positioning a distal end of the elongate medical device near a target region of the body;
rigidizing the elongate medical device;
inserting a liner insert tube into an expandible working channel of the tube, so that the expandable working channel expands to accommodate the liner insert tube; and
inserting a working tool through the liner insert tube and out of a distal end of the liner insert tube.
